(19) United States
(12) Patent Application Publication
(10) Pub. No.: US 2022/0389082 A1
(43) Pub. Date: Dec. 8, 2022

(54) A TRUNCATED VON WILLEBRAND FACTOR (VWF) FOR INCREASING THE IN VITRO STABILITY OF COAGULATION FACTOR VIII

(71) Applicant: CSL BEHRING LENGNAU AG, Lengnau BE (CH)

(72) Inventors: Hubert METZNER, Marburg (DE); Uwe LIEBING, Coelbe (DE)

(73) Assignee: CSL BEHRING LENGNAU AG, Lengnau BE (CH)

(21) Appl. No.: 17/623,398

(22) PCT Filed: Jul. 3, 2020
(86) PCT No.: PCT/EP2020/068772

§ 371 Date: Dec. 28, 2021

(30) Foreign Application Priority Data
Jul. 4, 2019 (EP) 19184390.3

(51) Int. Cl. C07K 14/755

(52) U.S. Cl. CPC C07K 14/755; C07K 2319/31; C07K 2319/30

(57) ABSTRACT

The present invention relates to the use of a polypeptide comprising a truncated von Willebrand Factor (VWF) for increasing the in vitro stability of coagulation factor VIII (FVIII) in a composition comprising said FVIII and said polypeptide, wherein the molar ratio of the polypeptide to the FVIII in the composition is greater than 20.

### FIELD OF THE INVENTION

The present invention relates to methods for improving the in vitro stability of coagulation factor VIII (FVIII), particularly, to the use of a polypeptide comprising a truncated von Willebrand Factor (VWF) for increasing the in vitro stability of coagulation factor VIII (FVIII).

### BACKGROUND OF THE INVENTION

Factor VIII (FVIII) is a protein found in blood plasma, which acts as a cofactor in the cascade of reactions leading to blood coagulation. A deficiency in the amount of FVIII activity in the blood results in the clotting disorder known as hemophilia A, an inherited condition primarily affecting males. Hemophilia A is currently treated with therapeutic preparations of FVIII derived from human plasma or manufactured using recombinant DNA technology. In general, such preparations are administered either in response to a bleeding episode (on-demand therapy) or at frequent, regular intervals to prevent uncontrolled bleeding (prophylaxis).

FVIII is known to be relatively unstable in therapeutic preparations. In blood plasma, FVIII is usually complexed with another plasma protein, von Willebrand factor (VWF), which is believed to protect FVIII from premature degradation. Currently marketed FVIII preparations often rely on the use of albumin and/or native VWF to stabilize FVIII during the manufacturing process and during storage.

Several attempts to formulate FVIII without albumin or native VWF (or with relatively low levels of these excipients) have been described. For example, U.S. Pat. No. 5,565,427 (EP 0 508 194) describes FVIII preparations which contain particular combinations of detergent and amino acids, specifically arginine and glycine, in addition to excipients such as sodium chloride and sucrose.

U.S. Pat. No. 5,763,401 (EP 0 818 204) also describes a therapeutic FVIII formulation comprising 15-60 mM sucrose, up to 50 mM NaCl, up to 5 mM calcium chloride, 65-400 mM glycine, and up to 50 mM histidine.

U.S. Pat. No. 5,733,873 (EP 627 924) discloses formulations which include between 0.01-1 mg/ml of a surfactant. Other attempts to use low or high concentrations of sodium chloride have also been described. U.S. Pat. No. 4,877,608 (EP 0 315 968) discloses formulations with relatively low concentrations of sodium chloride, namely 0.5 mM to 15 mM NaCl. On the other hand, U.S. Pat. No. 5,605,884 (EP 0 314 095) teaches the use of formulations with relatively high concentrations of sodium chloride.

Further FVIII formulations are disclosed in WO 2010/054238, EP 1 712 223, WO 2000/48635, WO 96/30041, WO 96/22107, WO 2011/027152, EP 2 361 613, EP 0 410 207, EP 0 511 234, U.S. Pat. No. 5,565,427, EP 0 638 091, EP 0 871 476, EP 0 819 010, U.S. Pat. No. 5,874,408, US 2005/0256038, US 2008/0064856, WO 2005/058283, WO 2012/037530 and WO 2014/026954.

VWF-derived polypeptides, in particular VWF fragments, have been described to improve bioavailability of FVIII in vivo. WO 2013/106787 A1 is directed at chimeric proteins comprising a FVIII protein and certain VWF fragments. Those chimeric hetero-dimers of FVIII and VWF-fragment do have a fixed molar ratio of VWF to FVIII of 1:1.

WO 2014/198699 A2 and WO 2013/083858 A2 describe VWF fragments and their use in the treatment of hemophilia. It was found that bioavailability of FVIIIs may be significantly improved upon extravascular co-administration with similar molar amounts of VWF fragments. WO 2018/087271 A1 and WO 2016/188907 A1 describe truncated VWF polypeptides for the treatment or prophylaxis of hemophilia. WO 2016/000039 A1, WO 2017/117630 A1 and WO 2017/117631 A1 describe modified VWF polypeptides capable of binding to FVIII.

WO 2011/060242 A2 discloses fusion polypeptides comprising certain VWF fragments and an antibody Fc region proposing specific molar ratios of VWF fragment over FVIII of up to 10:1. In addition, no in vivo data are presented with regard to said Fc-fusion constructs.

Yee et al. (2014) Blood 124(3):445-452 found that a VWF fragment containing the D′D3 domains fused to the Fc portion of immunoglobulin G1 is sufficient to stabilize endogenous Factor VIII in VWF-deficient mice. Hence, in VWF-deficient mice the endogenous expression rate of FVIII was increased or the elimination rate of endogenously expressed FVIII was reduced. However, although a VWF D′D3-Fc fusion protein exhibited markedly prolonged survival when transfused into FVIII-deficient mice, the VWF D′D3-Fc fusion protein did not prolong the survival of co-transfused FVIII.

WO 2015/185758 A2 describes compositions comprising a complex of FVIII and one or more VWF peptides, wherein the VWF peptides comprise at least the amino acids 764 to 1035 and 1691 to 1905 of human VWF (UniProtKB-P04275) but not amino acids 2255 to 2645 of human VWF. A VWF fragment consisting of amino acids 764-2128 of human VWF (“fragment III”) was prepared by digestion of plasma derived VWF with S. aureus V-8 protease. This fragment bound to collagen III and heparin. Fragment III stabilized rFVIII in solution when added in a five-fold molar excess, calculated on the basis of the fragment III monomer subunit. WO 2015/185758 A2 does not show a stabilizing effect for VWF peptides lacking amino acids 1691 to 1905. WO 2015/185758 A2 does not disclose any ratios of VWF peptide to FVIII greater than 20.

There is a continued need for means and methods to stabilize FVIII preparations in vitro.

### SUMMARY OF THE INVENTION

The inventors of this application surprisingly found that VWF fragments comprising the D′D3 domain of VWF increased the stability of FVIII in vitro, at a molar ratio of VWF fragment to FVIII of greater than 20.

The present invention therefore relates to the subject matter defined in the following items [1] to [80]:

[1] The use of a polypeptide comprising a truncated von Willebrand Factor (VWF) for increasing the in vitro stability of coagulation factor VIII (FVIII) in a composition comprising said FVIII and said polypeptide, wherein the molar ratio of the polypeptide to the FVIII in the composition is greater than 20.

[2] The use according to item [1], wherein said polypeptide increases the storage stability of said FVIII.

[3]. The use according to item [1] or [2], wherein the composition does not comprise a protease.

[4] The use of any one of the preceding items, wherein the composition does not comprise wild-type VWF.

[5] The use of any one of the preceding items, wherein said FVIII is recombinantly produced FVIII or plasma derived FVIII.

[6] The use of any one of the preceding items, wherein said molar ratio is at least 50.

[7] The use of any one of the preceding items, wherein said molar ratio is greater than 50.

[8] The use of any one of the preceding items, wherein said molar ratio is at least 60.

[9] The use of any one of the preceding items, wherein said molar ratio is at least 75.

[10] The use of any one of the preceding items, wherein said molar ratio is at least 100.

[11] The use of any one of the preceding items, wherein said molar ratio is at least 200.

[12] The use of any one of the preceding items, wherein said molar ratio is at least 300.

[13] The use of any one of the preceding items, wherein said molar ratio is at least 400.

[14] The use of any one of the preceding items, wherein said molar ratio is at least 500.

[15] The use of any one of the preceding items, wherein said molar ratio is at least 600.

[16] The use of any one of the preceding items, wherein said molar ratio is at least 700.

[17] The use of any one of the preceding items, wherein said molar ratio is less than 10,000.

[18] The use of any one of the preceding items, wherein said molar ratio is from greater than 20 to less than 10,000.

[19] The use of any one of the preceding items, wherein said molar ratio is from about 25 to about 9,000.

[20] The use of any one of the preceding items, wherein said molar ratio is from about 50 to about 7,500.

[21] The use of any one of the preceding items, wherein said molar ratio is from greater than 50 to about 6,000.

[22] The use of any one of the preceding items, wherein said molar ratio is from about 60 to about 5,000.

[23] The use of any one of the preceding items, wherein said molar ratio is from greater than 75 to about 4,000.

[24] The use of any one of the preceding items, wherein said molar ratio is from about 100 to about 3,000.

[25] The use of any one of the preceding items, wherein said molar ratio is from about 200 to about 2,500.

[26] The use of any one of the preceding items, wherein said molar ratio is from about 300 to about 2,000.

[27] The use of any one of the preceding items, wherein said molar ratio is from about 400 to about 1,750.

[28] The use of any one of the preceding items, wherein said molar ratio is from about 500 to about 1,500.

[29] The use of any one of the preceding items, wherein said molar ratio is from about 600 to about 1,250.

[30] The use of any one of the preceding items, wherein said molar ratio is from about 700 to about 1,000.

[31] The use of any one of the preceding items, wherein said use comprises adding a more than 20-fold molar excess of said polypeptide to said FVIII, thereby stabilizing said FVIII.

[32] The use of item [31], wherein said molar excess is at least 25-fold, or at least 50-fold, or greater than 50-fold, or at least 60-fold, or at least 75-fold, or at least 100-fold, or at least 200-fold, or at least 300-fold, or at least 400-fold, or at least 500-fold, or at least 600-fold, or at least 700-fold.

[33] The use of item [31] or [32], wherein said molar excess is from greater than 20-fold to less than 10,000-fold, or from 25-fold to 9,000-fold, or from 50-fold to 7,500-fold, or from 60-fold to 5,000-fold, or from 75-fold to 4,000-fold, or from 100-fold to 3,000-fold, or from 200-fold to 2,500-fold, or from 300-fold to 2,000-fold, or from 400-fold to 1,750-fold, or from 500-fold to 1,500-fold, or from 600-fold to 1,250-fold, or from 700-fold to 1,000-fold.

[34] The use of any one of the preceding items, wherein the yield of FVIII upon freeze-drying and reconstituting the composition comprising the FVIII and the polypeptide is greater than the yield of FVIII upon freeze-drying and reconstituting a control composition lacking said polypeptide.

[35] The use of item [34], wherein the freeze-dried composition is reconstituted shortly after freeze-drying.

[36] The use of item [34] or [35], wherein the loss in FVIII activity over the freeze-drying process is less than 13%.

[37] The use of item [34] or [35], wherein the loss in FVIII activity over the freeze-drying process is 11% or less.

[38] The use of item [34] or [35], wherein the loss in FVIII activity over the freeze-drying process is 10% or less.

[39] The use of item [34] or [35], wherein the loss in FVIII activity over the freeze-drying process is 8% or less.

[40] The use of item [34] or [35], wherein the loss in FVIII activity over the freeze-drying process is 5% or less.

[41] The use of item [34] or [35], wherein the loss in FVIII activity over the freeze-drying process is less than 3%.

[42] The use of item [34] or [35], wherein the loss in FVIII activity over the freeze-drying process is 2% or less.

[43] The use of any one of items [1] to [33], wherein the loss in FVIII activity during storage at 25° C. of a freeze-dried composition comprising the FVIII and the polypeptide is lower than that of a freeze-dried control composition lacking said polypeptide.

[44] The use of item [43], wherein said storage is for a period of 12 months.

[45] The use of item [43], wherein the loss in FVIII activity during said storage at 25° C. is less than 20%, preferably less than 18%, more preferably less than 16%.

[46] The use of item [43], wherein said storage is for a period of 24 months.

[47] The use of item [43], wherein the loss in FVIII activity during said storage at 25° C. is less than 30%, preferably less than 20%.

[48] The use according to any one of items [1] to [33], wherein the FVIII activity in a liquid composition comprising said polypeptide and said FVIII after storage at 25° C. for at least one week is greater than that of a control composition lacking said polypeptide.

[49] The use of item [48], wherein the loss in FVIII activity during storage at 25° C. for one week is less than 10%.

[50] The use of item [48], wherein the loss in FVIII activity during storage at 25° C. for four weeks is less than 20%, or less than 15%.

[51] The recombinant polypeptide for use according to any one of the preceding embodiments, wherein the truncated VWF is a human truncated VWF.

[52] The use according to any one of the preceding items, wherein the truncated VWF comprises an amino acid sequence having a sequence identity of at least 90% to amino acids 776 to 805 of SEQ ID NO:4, preferably comprises an amino acid sequence having a sequence identity of at least 90% to amino acids 764 to 1242 of SEQ ID NO:4.

[53] The use according to any one of the preceding items, wherein the truncated VWF lacks amino acids 1243 to 2813 of SEQ ID NO:4.

[54] The use according to any one of the preceding items, wherein the truncated VWF consists either of (a) amino acids 764 to 1242 of SEQ ID NO:4, of (b) an amino acid sequence having a sequence identity of at least 90% to amino acids 764 to 1242 of SEQ ID NO:4, or of (c) a fragment of (a) or (b).

[55] The use according to any one of the preceding items, wherein said polypeptide binds to said FVIII with a dissociation constant K_{D} of 1 μM or less.

[56] The use according to any one of the preceding items, wherein said polypeptide binds to said FVIII with a dissociation constant K_{D} of 1 nM or less.

[57] The use according to any one of the preceding items, wherein said polypeptide binds to said FVIII with a dissociation constant K_{D} of 0.1 nM or less.

[58] The use according to any one of the preceding items, wherein said polypeptide comprises a half-life extending moiety (HLEM).

[59] The use according to item [58], wherein the HLEM is a heterologous amino acid sequence fused to the truncated VWF.

[60] The use according to item [59], wherein said heterologous amino acid sequence comprises or consists of a protein or peptide selected from the group consisting of transferrin and fragments thereof, the C-terminal peptide of human chorionic gonadotropin, an XTEN sequence, homo-amino acid repeats (HAP), proline-alanine-serine repeats (PAS), albumin and fragments thereof, afamin, alpha-fetoprotein, Vitamin D binding protein, polypeptides capable of binding under physiological conditions to albumin or immunoglobulin constant regions, polypeptides capable of binding to the neonatal Fc receptor (FcRn), particularly immunoglobulin constant regions and portions thereof, preferably the Fc portion of immunoglobulin, and combinations thereof.

[61] The use according to item [58], wherein the HLEM is conjugated to the polypeptide comprising the truncated VWF.

[62] The use according to item [61], wherein the HLEM is conjugated to the C-terminus of the polypeptide comprising the truncated VWF.

[63] The use according to item [61] or [62], wherein said HLEM is selected from the group consisting of hydroxyethyl starch (HES), polyethylene glycol (PEG), polysialic acids (PSAs), elastin-like polypeptides, heparosan polymers, hyaluronic acid and non-proteinaceous albumin binding ligands, e.g. fatty acid chains, and combinations thereof.

[64] The use according to item [58], wherein the HLEM is non-covalently linked to the polypeptide comprising the truncated VWF.

[65] The use according to any one of items [1] to [60], wherein the polypeptide comprising the truncated VWF does not comprise any HLEM conjugated to the polypeptide.

[66] The use according to any one of the preceding items, wherein said polypeptide is a glycoprotein comprising N-glycans, and wherein at least 50%, at least 75%, preferably at least 85% of said N-glycans comprise, on average, at least one sialic acid moiety.

[67] The use according to any one of the preceding items, wherein said polypeptide is present as a dimer or at least has a high proportion of dimers.

[68] The use according to item [67], wherein at least 50%, or at least 70%, or at least 80%, or at least 90%, or at least 95% of said polypeptide is present as a dimer.

[69] The use according to item [67] or [68], wherein said dimer is a homodimer, and the two monomers forming the dimer are covalently linked to each other via at least one or more disulfide bridges formed by cysteine residues within the truncated VWF.

[70] The use according to item [69], wherein the cysteine residues forming the one or more disulfide bridges is/are selected from the group consisting of Cys-1099, Cys-1142, Cys-1222, Cys-1225, Cys-1227 and combinations thereof, preferably Cys-1099 and Cys-1142, wherein the amino acid numbering refers to SEQ ID NO:4.

[71] The use according to any one of items [67] to [70], wherein the affinity of said dimer to FVIII is greater than the affinity of a monomeric polypeptide to FVIII, said monomeric polypeptide having the same amino acid sequence as a monomeric subunit of the dimeric polypeptide.

[72] The use according to any one of items [67] to [71], wherein the ratio dimer:monomer of the polypeptide is at least 1.5, preferably at least 2, more preferably at least 2.5 or at least 3, or at least 4, or at least 5, or at least 10, or at least 20; or wherein the polypeptide does not comprise monomer and/or multimer forms of the polypeptide; or wherein the polypeptide is essentially free of monomer and/or multimer forms of the polypeptide.

[73] The use according to any one of items [67] to [72], wherein the dimeric polypeptide has a FVIII binding affinity characterized by a dissociation constant K_{D} of less than 1 μM, preferably less than 1 nM, more preferably less than 500 μM, less than 200 μM, less than 100 μM, less than 90 μM or less than 80 μM.

[74] The use according to item [73], wherein the K_{D} ranges from 0.1 μM to 500 μM, from 0.5 μM to 200 μM, from 0.75 μM to 100 μM or most preferred from 1 μM to 80 μM.

[75] The use according to any one of the preceding items, wherein said polypeptide comprises at least one amino acid substitution as compared to the amino acid sequence of wild-type VWF, wherein the binding affinity of such a modified polypeptide to FVIII is preferably being further increased by introduction of said at least one substitution compared to the binding affinity of a reference polypeptide which has the same amino acid sequence except for said modifications.

[76] The use according to item [75], wherein the at least one substitution is selected from the group of combinations consisting of S764G/S766Y, S764P/S7661, S764P/S766M, S764V/S766Y, S764E/S766Y, S764Y/S766Y, S764L/S766Y, S764P/S766W, S766W/S806A, S766Y/P769K, S766Y/P769N, S766Y/P769R, S764P/S766L, and S764E/S766Y/V1083A, referring to the sequence of SEQ ID NO:4 with regard to the amino acid numbering.

[77] The use according to item [76], wherein said at least one substitution is either the combination S764E/S766Y or S764E/S766Y/V1083A.

[78] The use according to any one of the preceding items, wherein the composition is a formulation.

[79] The use according to item [78], wherein the formulation is suitable for the treatment or prophylaxis of a blood coagulation disorder.

[80] The use according to item [79], wherein the blood coagulation disorder is hemophilia A.

### DETAILED DESCRIPTION

The present invention relates to the use of a polypeptide comprising a truncated von Willebrand Factor (VWF) for increasing the in vitro stability of coagulation factor VIII (FVIII) in a composition comprising said FVIII and said polypeptide, wherein the molar ratio of the polypeptide to the FVIII in the composition is greater than 20.

The polypeptide comprising a truncated von Willebrand Factor (VWF) will be referred to herein as “polypeptide of the invention”. The polypeptide of the invention preferably comprises a half-life extending moiety.

Ratios

As described in more detail below, the polypeptide of the invention may be a monomer, a dimer, or a mixture thereof. Any molar ratios according to the invention refer to a ratio of the molar concentration of the monomeric subunit of the polypeptide of the invention, whether actually present as monomer, as dimer, or as oligomer. Ratios are formed over the molar concentration of the co-formulated FVIII. Any ratios of the polypeptide of the invention over FVIII in this application refer to the amount of monomer subunits (in mole) comprised in the polypeptide of the invention, which is preferably present as a dimer, divided by the amount of FVIII (in mole), unless indicated otherwise. By way of non-limiting example the co-formulation of 100 μM of a monomeric polypeptide of the invention with 1 μM of FVIII means a ratio of 100. The same ratio of 100 is obtained if 50 μM of a dimeric polypeptide of the invention are co-formulated with 1 μM of FVIII.

The molar ratio of the polypeptide of the invention to FVIII is above 20, or at least 25, or at least 50, or greater than 50, more preferably the ratio is at least 60, or at least 75, or at least 100, or greater than 100, or at least 200, most preferably at least 300, or at least 400, or at least 500, or at least 600, or at least 700, or at least 800, or at least 900, or at least 1,000, or at least 1,100, or at least 1,200, or at least 1,300, or at least 1,400, or at least 1,500, or at least 1,600, or at least 1,700, or at least 1,800, or at least 1,900, or at least 2,000, or at least 2,500, or at least 3,000 or at least 5,000, or at least 8,000 or up to 10,000. The molar ratio of the polypeptide of the invention to FVIII may according to certain embodiments not exceed a ratio of 10,000, a ratio of 5,000, a ratio of 2,500 or a ratio of 2,000.

The molar ratio of the polypeptide of the invention to FVIII may range from above 20 to 10,000, or from above 50 to 5,000, or from above 50 to 4,000, or from above 50 to 3,000, or from above 50 to 2,000, or from above 50 to 1,000. Preferably, the molar ratio of the polypeptide of the invention to FVIII ranges from 50 to 2,500, or from 75 to 2,000, or from 100 to 1,500, or from 150 to 1,000.

The Truncated VWF

The term “von Willebrand Factor” (VWF) as used herein includes naturally occurring (native) VWF, but also variants thereof retaining at least the FVIII binding activity of naturally occurring VWF, e.g. sequence variants where one or more residues have been inserted, deleted or substituted. The FVIII binding activity is determined by a FVIII-VWF binding assay as described in Example 2.

A preferred VWF in accordance with this invention is human VWF represented by the amino acid sequence shown in SEQ ID NO:4. The cDNA encoding SEQ ID NO:4 is shown in SEQ ID NO:3.

The gene encoding human native VWF is transcribed into a 9 kb mRNA which is translated into a pre-propolypeptide of 2813 amino acids with an estimated molecular weight of 310,000 Da. The pre-propolypeptide contains an N-terminal 22 amino acids signal peptide, followed by a 741 amino acid pro-polypeptide (amino acids 23-763 of SEQ ID NO:4) and the mature subunit (amino acids 764-2813 of SEQ ID NO:4). Cleavage of the 741 amino acids propolypeptide from the N-terminus results in mature VWF consisting of 2050 amino acids. The amino acid sequence of the human native VWF pre-propolypeptide is shown in SEQ ID NO:4. Unless indicated otherwise, the amino acid numbering of VWF residues in this application refers to SEQ ID NO:4, even if the VWF molecule, in particular a truncated VWF, does not comprise all residues of SEQ ID NO:4.

The propolypeptide of native VWF comprises multiple domains. Different domain annotations can be found in the literature (see, e.g. Zhou et al. (2012) Blood 120(2): 449-458). The following domain annotation of native pre-propolypeptide of VWF is applied in this application: D1-D2-D′-D3-A1-A2-A3-D4-C1-C2-C3-C4-C5-C6-CK

With reference to SEQ ID NO:4, the D′ domain consists of amino acids 764-865; and the D3 domain consists of amino acids 866-1242.

The feature “truncated” in terms of the present invention means that the polypeptide does not comprise the entire amino acid sequence of mature VWF (e.g. amino acids 764-2813 of SEQ ID NO:4). The truncated VWF does not comprise all amino acids 764-2813 of SEQ ID NO:4 but typically only a fragment thereof. A truncated VWF may also be referred to as a VWF fragment, or in the plural as VWF fragments.

Typically, the truncated VWF is capable of binding to a Factor VIII. Preferably, the truncated VWF is capable of binding to the mature form of human native Factor VIII. In another embodiment, the truncated VWF is capable of binding to a recombinant FVIII, e.g. to a FVIII as described herein, such as the single-chain Factor VIII consisting of the amino acid sequence SEQ ID NO:5. Binding of the truncated VWF to Factor VIII can be determined by a FVIII-VWF binding assay as described in Example 2.

The truncated VWF of the present invention preferably comprises or consists of an amino acid sequence having a sequence identity of at least 90% to amino acids 776 to 805 of SEQ ID NO:4 and is capable of binding to FVIII. In preferred embodiments the truncated VWF comprises or consists of an amino acid sequence having a sequence identity of at least 95%, at least 96%, at least 97%, at least 98%, or at least 99%, to amino acids 776 to 805 of SEQ ID NO:4 and is capable of binding to FVIII. In one embodiment, the truncated VWF comprises or consists of amino acids 776 to 805 of SEQ ID NO:4. Unless indicated otherwise herein, sequence identities are determined over the entire length of the reference sequence (e.g. amino acids 776 to 805 of SEQ ID NO:4).

The truncated VWF of the present invention preferably comprises or consists of an amino acid sequence having a sequence identity of at least 90% to amino acids 766 to 864 of SEQ ID NO:4 and is capable of binding to FVIII. In preferred embodiments the truncated VWF comprises or consists of an amino acid sequence having a sequence identity of at least 95%, at least 96%, at least 97%, at least 98%, or at least 99%, to amino acids 766 to 864 of SEQ ID NO:4 and is capable of binding to FVIII. In one embodiment, the truncated VWF comprises or consists of amino acids 766 to 864 of SEQ ID NO:4.

In another preferred embodiment, the truncated VWF consists of (a) an amino acid sequence having a sequence identity of at least 90% to amino acids 764 to 1242 of SEQ ID NO:4, or (b) a fragment thereof, provided that the truncated VWF is still capable of binding to FVIII. More preferably, the truncated VWF consists of (a) an amino acid sequence having a sequence identity of at least 95%, at least 96%, at least 97%, at least 98%, or at least 99%, to amino acids 764 to 1242 of SEQ ID NO:4, or (b) a fragment thereof, provided that the truncated VWF is still capable of binding to FVIII. In one embodiment, the truncated VWF consists of (a) amino acids 764 to 1242 of SEQ ID NO:4, or (b) a fragment thereof, provided that the truncated VWF is still capable of binding to FVIII.

As described in more detail below, the polypeptide of the invention may be prepared by a method which uses cells comprising a nucleic acid encoding the polypeptide comprising the truncated VWF. The nucleic acid is introduced into suitable host cells by techniques that are known per se.

In a preferred embodiment, the nucleic acid in the host cell encodes (a) an amino acid sequence having a sequence identity of at least 90% to amino acids 1 to 1242 of SEQ ID NO:4, or (b) a fragment thereof, provided that the truncated mature VWF is still capable of binding to FVIII. More preferably, the nucleic acid encodes (a) an amino acid sequence having a sequence identity of at least 95%, at least 96%, at least 97%, at least 98%, or at least 99%, to amino acids 1 to 1242 of SEQ ID NO:4, or (b) a fragment thereof, provided that the truncated VWF is still capable of binding to FVIII. In one embodiment, the nucleic acid encodes (a) amino acids 1 to 1242 of SEQ ID NO:4, or (b) a fragment thereof, provided that the truncated VWF is still capable of binding to FVIII. Especially if the polypeptide in accordance with this invention is a dimer, the nucleic acid will comprise a sequence also encoding amino acids 1 to 763 of VWF (e.g. SEQ ID NO:4), even if the truncated VWF in the polypeptide does not comprise amino acids 1 to 763 of VWF (e.g. SEQ ID NO:4).

The truncated VWF of the recombinant polypeptide of the invention according to a preferred embodiment may not comprise amino acid sequence 1 to 763 of VWF of SEQ ID NO:4.

According to further preferred embodiments, the truncated VWF comprises or consists of one of the following amino acid sequences, each referring to SEQ ID NO:4:

776-805; 766-805; 764-805; 776-810; 766-810; 764-810; 776-815; 766-815; 764-815; 776-820; 766-820; 764-820; 776-825; 766-825; 764-825; 776-830; 766-830; 764-830; 776-835; 766-835; 764-835; 776-840; 766-840; 764-840; 776-845; 766-845; 764-845; 776-850; 766-850; 764-850; 776-855; 766-855; 764-855; 776-860; 766-860; 764-860; 776-864; 766-864; 764-864; 776-865; 766-865; 764-865; 776-870; 766-870; 764-870; 776-875; 766-875; 764-875; 776-880; 766-880; 764-880; 776-885; 766-885; 764-885; 776-890; 766-890; 764-890; 776-895; 766-895; 764-895; 776-900; 766-900; 764-900; 776-905; 766-905; 764-905; 776-910; 766-910; 764-910; 776-915; 766-915; 764-915; 776-920; 766-920; 764-920; 776-925; 766-925; 764-925; 776-930; 766-930; 764-930; 776-935; 766-935; 764-935; 776-940; 766-940; 764-940; 776-945; 766-945; 764-945; 776-950; 766-950; 764-950; 776-955; 766-955; 764-955; 776-960; 766-960; 764-960; 776-965; 766-965; 764-965; 776-970; 766-970; 764-970; 776-975; 766-975; 764-975; 776-980; 766-980; 764-980; 776-985; 766-985; 764-985; 776-990; 766-990; 764-990; 776-995; 766-995; 764-995; 776-1000; 766-1000; 764-1000; 776-1005; 766-1005; 764-1005; 776-1010; 766-1010; 764-1010; 776-1015; 766-1015; 764-1015; 776-1020; 766-1020; 764-1020; 776-1025; 766-1025; 764-1025; 776-1030; 766-1030; 764-1030; 776-1035; 766-1035; 764-1035; 776-1040; 766-1040; 764-1040; 776-1045; 766-1045; 764-1045; 776-1050; 766-1050; 764-1050; 776-1055; 766-1055; 764-1055; 776-1060; 766-1060; 764-1060; 776-1065; 766-1065; 764-1065; 776-1070; 766-1070; 764-1070; 776-1075; 766-1075; 764-1075; 776-1080; 766-1080; 764-1080; 776-1085; 766-1085; 764-1085; 776-1090; 766-1090; 764-1090; 776-1095; 766-1095; 764-1095; 776-1100; 766-1100; 764-1100; 776-1105; 766-1105; 764-1105; 776-1110; 766-1110; 764-1110; 776-1115; 766-1115; 764-1115; 776-1120; 766-1120; 764-1120; 776-1125; 766-1125; 764-1125; 776-1130; 766-1130; 764-1130; 776-1135; 766-1135; 764-1135; 776-1140; 766-1140; 764-1140; 776-1145; 766-1145; 764-1145; 776-1150; 766-1150; 764-1150; 776-1155; 766-1155; 764-1155; 776-1160; 766-1160; 764-1160; 776-1165; 766-1165; 764-1165; 776-1170; 766-1170; 764-1170; 776-1175; 766-1175; 764-1175; 776-1180; 766-1180; 764-1180; 776-1185; 766-1185; 764-1185; 776-1190; 766-1190; 764-1190; 776-1195; 766-1195; 764-1195; 776-1200; 766-1200; 764-1200; 776-1205; 766-1205; 764-1205; 776-1210; 766-1210; 764-1210; 776-1215; 766-1215; 764-1215; 776-1220; 766-1220; 764-1220; 776-1225; 766-1225; 764-1225; 776-1230; 766-1230; 764-1230; 776-1235; 766-1235; 764-1235; 776-1240; 766-1240; 764-1240; 776-1242; 766-1242; 764-1242; 764-1464; 764-1250; 764-1041; 764-828; 764-865; 764-1045; 764-1035; 764-1128; 764-1198; 764-1268; 764-1261; 764-1264; 764-1459; 764-1463; 764-1464; 764-1683; 764-1873; 764-1482; 764-1479; 764-1672; and 764-1874.

In certain embodiments the truncated VWF has an internal deletion relative to mature wild type VWF. For example, the A1, A2, A3, D4, C1, C2, C3, C4, C5, C6, CK domains or combinations thereof may be deleted, and the D′ domain and/or the D3 domain is retained. According to further embodiments, the truncated VWF lacks one or more of the domains A1, A2, A3, D4, C1, C2, C3, C4, C5, C6 or CK. According to further embodiments, the truncated VWF lacks amino acids 1243 to 2813 of SEQ ID NO:4, i.e. the domains A1-A2-A3-D4-C1-C2-C3-C4-C5-C6-CK.

In further embodiments the truncated VWF does not comprise the binding sites for platelet glycoprotein Ibα (GPIbα), collagen and/or integrin αIIbβIII (RGDS sequence within the C1 domain). In other embodiments, the truncated VWF does not comprise the cleavage site (Tyr1605-Met1606) for ADAMTS13 which is located at the central A2 domain of VWF. In yet another embodiment, the truncated VWF does not comprise the binding sites for GPIbα, and/or does not comprise the binding site(s) for collagen, and/or does not comprise the binding site for integrin αIIbβIII, and/or it does not comprise the cleavage site (Tyr1605-Met1606) for ADAMTS13 which is located at the central A2 domain of VWF. In a preferred embodiment the truncated VWF does not comprise amino acids 1691 to 1905 of SEQ ID NO:4. In another preferred embodiment the truncated VWF does not comprise amino acids 1691 to 1905 of the amino acid sequence deposited as UniProtKB-P04275. In another preferred embodiment the truncated VWF does not comprise amino acids 1691 to 1905 of human VWF.

In one embodiment the polypeptide has a low affinity for platelets, said low affinity being characterized by a dissociation constant K_{D}>1 μM, preferentially K_{D}>10 μM, for binding of the polypeptide to GPIbα.

In another embodiment the polypeptide does not contain the VWF domains A1 and/or A3 or a part thereof and does have low or essentially no affinity for collagen type I and type III, said low or essentially no affinity being characterized by a dissociation constant K_{D}>1 μM, preferentially K_{D}>10 μM, for binding of the polypeptide to collagen type I and type III. Said polypeptide may, however, contain one or more copies of a peptide having, preferably consisting of, amino acids 1238 to 1268 of SEQ ID NO: 4 fused N- or C-terminally to the polypeptide.

In other embodiments the truncated VWF comprises or consists of an amino acid sequence that has a sequence identity of at least 90%, or at least 91%, or at least 92%, or at least 93%, or at least 94%, or at least 95%, or at least 96%, or at least 97%, or at least 98%, or at least 99%, to one of the amino acid sequences recited in the preceding paragraph, provided that the truncated VWF is capable of binding to FVIII.

A polypeptide of the invention is termed a “dimer” in the present invention if two monomers of the polypeptide of the invention are linked covalently. Preferably, the covalent bond is located within the truncated VWF portion of the polypeptide of the invention. Preferably, the two monomeric subunits are covalently linked via at least one disulfide bridge, e.g. by one, two, three or four disulfide bridges. The cysteine residues forming the at least one disulfide bridge are preferably located within the truncated VWF portion of the polypeptide of the invention. In one embodiment, these cysteine residues are Cys-1099, Cys-1142, Cys-1222, Cys-1225, or Cys-1227 or combinations thereof. Preferably, the dimeric polypeptide of the invention does not comprise any further covalent bond linking the monomers in addition to said covalent bond located within the truncated VWF portion of the polypeptide, in particular does not comprise any further covalent bond located within the HLEM or HLEP portion of the polypeptide. According to alternative embodiments, however, the dimeric polypeptide of the invention may comprise a covalent bond located in the HLEM or HLEP portion of the polypeptide linking the monomers.

The dimer is preferably a homo-dimer, whereby each monomer comprises preferably a HLEM or HLEP as disclosed herein. If the polypeptide of the invention is a dimer, the truncated VWF preferably comprises or consists of two polypeptides each with an amino acid sequence having a sequence identity of at least 90% to amino acids 764 to 1099, amino acids 764 to 1142, amino acids 764 to 1222, amino acids 764 to 1225, amino acids 764 to 1227 or amino acids 764 to 1242 of SEQ ID NO:4 and is capable of binding to FVIII. In preferred embodiments the truncated VWF comprises or consists of an amino acid sequence having a sequence identity of at least 95%, at least 96%, at least 97%, at least 98%, or at least 99%, to amino acids 764 to 1099, amino acids 764 to 1142, amino acids 764 to 1222, amino acids 764 to 1225, amino acids 764 to 1227 or amino acids 764 to 1242 of SEQ ID NO:4 and is capable of binding to FVIII. Most preferably, the truncated VWF comprises or consists of amino acids 764 to 1099, amino acids 764 to 1142, amino acids 764 to 1222, amino acids 764 to 1225, amino acids 764 to 1227 or amino acids 764 to 1242 of SEQ ID NO:4.

The truncated VWF may be any one of the VWF fragments disclosed in WO 2013/106787 A1, WO 2014/198699 A2, WO 2011/060242 A2 or WO 2013/093760 A2, the disclosure of which is incorporated herein by reference.

According to further preferred embodiments, the truncated VWF as disclosed above may comprise at least one of the amino acid substitutions as disclosed in WO 2016/000039 A1. Those modified versions of the truncated VWF comprise at least one amino acid substitution within its D′ domain, as compared to the amino acid sequence of the D′ domain of wild-type VWF according to SEQ ID NO: 4. The amino acid sequence of the modified versions of the truncated VWF can have one or more amino acid substitutions relative to the respective wild type sequence. The amino acid sequence of the D′ domain of the modified truncated VWF preferably has one or 2 amino acid substitutions relative to the D′ domain of SEQ ID NO:4. It is preferred that S at position 764 of SEQ ID NO:4, corresponding to position 1 of SEQ ID NO:2, is substituted with an amino acid selected from the group consisting of G, P, V, E, Y, A and L. It is also preferred that S at position 766 of SEQ ID NO:4, corresponding to position 3 of SEQ ID NO:2 is substituted with an amino acid selected from the group consisting of Y, I, M, V, F, H, R and W. Preferred combinations of substitutions include S764G/S766Y, S764P/S7661, S764P/S766M, S764V/S766Y, S764E/S766Y, S764Y/S766Y, S764L/S766Y, S764P/S766W, S766W/S806A, S766Y/P769K, S766Y/P769N, S766Y/P769R and S764P/S766L, referring to the sequence of SEQ ID NO:4. The binding affinity of the polypeptide of the present invention to FVIII may be further increased by introduction of said substitutions compared to the binding affinity of a reference polypeptide which has the same amino acid sequence except for said modifications. Said substitutions within the truncated VWF may contribute to increase the half-life of co-administered FVIII, or the stability of co-formulated FVIII.

Half-Life Extending Moiety (HLEM)

In addition to the truncated VWF, the polypeptide of the invention may in certain preferred embodiments further comprise a half-life extending moiety. The half-life-extending moiety may be a heterologous amino acid sequence fused to the truncated VWF. Alternatively, the half-life-extending moiety may be chemically conjugated to the polypeptide comprising the truncated VWF by a covalent bond that may be different from a peptide bond.

In certain embodiments of the invention, the half-life of the polypeptide of the invention is extended by chemical modification, e.g. attachment of a half-life extending moiety such as polyethylene glycol (PEGylation), glycosylated PEG, hydroxyl ethyl starch (HESylation), polysialic acids, elastin-like polypeptides, heparosan polymers or hyaluronic acid. In another embodiment, the polypeptide of the invention is conjugated to a HLEM such as albumin via a chemical linker. The principle of this conjugation technology has been described in an exemplary manner by Conjuchem LLC (see, e.g., U.S. Pat. No. 7,256,253).

In other embodiments, the half-life-extending moiety is a half-life enhancing polypeptide (HLEP). Preferably, the HLEP is an albumin or a fragment thereof. The N-terminus of the albumin may be fused to the C-terminus of the truncated VWF. Alternatively, the C-terminus of the albumin may be fused to the N-terminus of the truncated VWF. One or more HLEPs may be fused to the N- or C-terminal part of VWF provided that they do not to interfere with or abolish the binding capability of the truncated VWF to FVIII.

The recombinant polypeptide further comprises preferably a covalent bond positioned between the truncated VWF and the HLEM, or a linker sequence positioned between the truncated VWF and the HLEM.

Said linker sequence may be a peptidic linker consisting of one or more amino acids, in particular of 1 to 50, 1 to 30, 1 to 20, 1 to 15, 1 to 10, 1 to 5 or 1 to 3 (e.g. 1, 2 or 3) amino acids and which may be equal or different from each other. Preferably, the linker sequence is not present at the corresponding position in the wild-type VWF. Preferred amino acids present in said linker sequence include Gly and Ser. The linker sequence should be non-immunogenic. Preferred linkers may be comprised of alternating glycine and serine residues. Suitable linkers are described for example in WO2007/090584.

In another embodiment of the invention the peptidic linker between the truncated VWF moiety and the HLEP consists of peptide sequences, which serve as natural interdomain linkers or sequences in human proteins. Preferably, such peptide sequences in their natural environment are located close to the protein surface and are accessible to the immune system so that one can assume a natural tolerance against this sequence. Examples are given in WO 2007/090584. Cleavable linker sequences are described, e.g., in WO 2013/120939 A1.

In a preferred embodiment of the recombinant polypeptide the linker between the truncated VWF and the HLEP is a glycine/serine peptidic linker having or consisting of amino acid sequence 480-510 of SEQ ID NO:2.

In one embodiment the polypeptide has the following structure:

tVWF−L1−H, [formula 1]

Wherein tVWF is the truncated VWF, L1 is a chemical bond or a linker sequence, and H is a HLEM, in particular a HLEP.

L1 may be a chemical bond or a linker sequence consisting of one or more amino acids, e.g. of 1 to 50, 1 to 30, 1 to 20, 1 to 15, 1 to 10, 1 to 5 or 1 to 3 (e.g. 1, 2 or 3) amino acids and which may be equal or different from each other. Usually, the linker sequences are not present at the corresponding position in the wild-type VWF. Examples of suitable amino acids present in L1 include Gly and Ser. The linker should be non-immunogenic and may be a non-cleavable or cleavable linker. Non-cleavable linkers may be comprised of alternating glycine and serine residues as exemplified in WO 2007/090584 A1. In another embodiment of the invention the peptidic linker between the truncated VWF moiety and the albumin moiety consists of peptide sequences, which serve as natural interdomain linkers or sequences in human proteins. Preferably such peptide sequences in their natural environment are located close to the protein surface and are accessible to the immune system so that one can assume a natural tolerance against this sequence. Examples are given in WO2007/090584. Cleavable linker sequences are described, e.g., in WO 2013/120939 A1.

Preferred HLEP sequences are described infra. Likewise encompassed by the invention are fusions to the exact “N-terminal amino acid” or to the exact “C-terminal amino acid” of the respective HLEP, or fusions to the “N-terminal part” or “C-terminal part” of the respective HLEP, which includes N-terminal deletions of one or more amino acids of the HLEP. The polypeptide may comprise more than one HLEP sequence, e.g. two or three HLEP sequences. These multiple HLEP sequences may be fused to the C-terminal part of VWF in tandem, e.g. as successive repeats.

Half-Life Enhancing Polypeptides (HLEPs)

Preferably, the half-life extending moiety is a half-life extending polypeptide (HLEP). More preferably the HLEP is selected from the group consisting of albumin, a member of the albumin-family or fragments thereof, solvated random chains with large hydrodynamic volume (e.g. XTEN (Schellenberger et al. 2009; Nature Biotechnol. 27:1186-1190), homo-amino acid repeats (HAP) or proline-alanine-serine repeats (PAS), afamin, alpha-fetoprotein, Vitamin D binding protein, transferrin or variants or fragments thereof, carboxyl-terminal peptide (CTP) of human chorionic gonadotropin-13 subunit, a polypeptide capable of binding to the neonatal Fc receptor (FcRn), in particular an immunoglobulin constant region and portions thereof, e.g. the Fc fragment, polypeptides or lipids capable of binding under physiological conditions to albumin, to a member of the albumin-family or to fragments thereof or to an immunoglobulin constant region or portions thereof. The immunoglobulin constant region or portions thereof is preferably an Fc fragment of immunoglobulin G1, an Fc fragment of immunoglobulin G2 or an Fc fragment of immunoglobulin A. The HLEP may in addition or alternatively comprise one or more copies of a peptide having, preferably consisting of amino acids 1238 to 1268 of SEQ ID NO: 4. Said peptide comprises preferably multiple O-glycosylated amino acids.

A half-life enhancing polypeptide as used herein may be a full-length half-life-enhancing protein described herein or one or more fragments thereof that are capable of stabilizing or prolonging the therapeutic activity or the biological activity of the coagulation factor, in particular of increasing the in vivo half-life of the polypeptide of the invention. Such fragments may be of 10 or more amino acids in length or may include at least about 15, at least about 20, at least about 25, at least about 30, at least about 50, at least about 100, or more contiguous amino acids from the HLEP sequence or may include part or all of specific domains of the respective HLEP, as long as the HLEP fragment provides a functional half-life extension of at least 25% compared to the respective polypeptide without the HLEP.

The HLEP portion of the polypeptide of the invention may be a variant of a wild type HLEP. The term “variants” includes insertions, deletions and substitutions, either conservative or non-conservative, where such changes do not substantially alter the FVIII-binding activity of the truncated VWF.

In particular, the proposed truncated VWF-HLEP fusion constructs of the invention may include naturally occurring polymorphic variants of HLEPs and fragments of HLEPs. The HLEP may be derived from any vertebrate, especially any mammal, for example human, monkey, cow, sheep, or pig. Non-mammalian HLEPs include, but are not limited to, hen and salmon.

According to certain embodiments of the present disclosure, the HLEM, in particular a HLEP, portion of the recombinant polypeptide of the invention may be specified with the alternative term “FP”. Preferably, the term “FP” represents a human albumin.

According to certain preferred embodiments, the recombinant polypeptide is a fusion protein. A fusion protein in terms of the present invention is a protein created by in-frame joining of at least two DNA sequences encoding the truncated VWF as well as the HLEP. The skilled person understands that translation of the fusion protein DNA sequence will result in a single protein sequence. As a result of an in frame insertion of a DNA sequence encoding a peptidic linker according to a further preferred embodiment, a fusion protein comprising the truncated VWF, a suitable linker and the HLEP may be obtained.

According to some embodiments, the co-formulated FVIII does neither comprise any of the herein described HLEM or HLEP structures. According to certain other embodiments, the co-formulated FVIII may comprise at least one of the herein described HLEM or HLEP structures.

Albumin as HLEP

The terms, “human serum albumin” (HSA) and “human albumin” (HA) are used interchangeably in this application. The terms “albumin” and “serum albumin” are broader, and encompass human serum albumin (and fragments and variants thereof) as well as albumin from other species (and fragments and variants thereof).

As used herein, “albumin” refers collectively to albumin polypeptide or amino acid sequence, or an albumin fragment or variant, having one or more functional properties (e.g., biological functions) of albumin. In particular, “albumin” refers to human albumin or fragments thereof, especially the mature form of human albumin as shown in SEQ ID NO:6 herein or albumin from other vertebrates or fragments thereof, or analogs or variants of these molecules or fragments thereof.

According to certain embodiments of the present disclosure the alternative term ‘FP’ is used to identify the HLEP, in particular to define albumin as HLEP.

In particular, the proposed polypeptides of the invention may include naturally occurring polymorphic variants of human albumin and fragments of human albumin. Generally speaking, an albumin fragment or variant will be at least 10, preferably at least 40, most preferably more than 70 amino acids long.

Preferred embodiments of the invention include albumin variants used as a HLEP of the polypeptide of the invention with enhanced binding to the FcRn receptor. Such albumin variants may lead to a longer plasma half-life of a truncated VWF albumin variant fusion protein as compared to a truncated VWF fusion with a wild-type albumin.

The albumin portion of the polypeptides of the invention may comprise at least one subdomain or domain of HA or conservative modifications thereof.

Immunoglobulins as HLEPs

Immunoglobulin G (IgG) constant regions (Fc) are known in the art to increase the half-life of therapeutic proteins (Dumont J A et al. 2006. BioDrugs 20:151-160). The IgG constant region of the heavy chain consists of 3 domains (CH1-CH3) and a hinge region. The immunoglobulin sequence may be derived from any mammal, or from subclasses IgG1, IgG2, IgG3 or IgG4, respectively. IgG and IgG fragments without an antigen-binding domain may also be used as HLEPs. The therapeutic polypeptide portion is connected to the IgG or the IgG fragments preferably via the hinge region of the antibody or a peptidic linker, which may even be cleavable. Several patents and patent applications describe the fusion of therapeutic proteins to immunoglobulin constant regions to enhance the therapeutic proteins' in vivo half-lives. US 2004/0087778 and WO 2005/001025 describe fusion proteins of Fc domains or at least portions of immunoglobulin constant regions with biologically active peptides that increase the half-life of the peptide, which otherwise would be quickly eliminated in vivo. Fc-IFN-β fusion proteins were described that achieved enhanced biological activity, prolonged circulating half-life and greater solubility (WO 2006/000448 A2). Fc-EPO proteins with a prolonged serum half-life and increased in vivo potency were disclosed (WO 2005/063808 A1) as well as Fc fusions with G-CSF (WO 2003/076567 A2), glucagon-like peptide-1 (WO 2005/000892 A2), clotting factors (WO 2004/101740 A2) and interleukin-10 (U.S. Pat. No. 6,403,077), all with half-life enhancing properties.

Various HLEPs which can be used in accordance with this invention are described in detail in WO 2013/120939 A1.

N-Glycans and Sialylation of the Polypeptide of the Invention

The polypeptide of the invention preferably comprises N-glycans, and at least 50%, preferably at least 75%, more preferably at least 85%, even more preferably at least 90% of said N-glycans comprise, on average, at least one sialic acid moiety. In preferred embodiments, at least 91%, at least 92%, at least 93%, at least 94%, at least 95%, at least 96%, at least 97%, at least 98%, or at least 99%, of said N-glycans comprise, on average, at least one sialic acid moiety.

When using mammalian cells for expression of the polypeptide of the invention, this often contains α-2,3-linked sialyl groups. But furthermore, the polypeptide of the invention may comprise N-glycans with α-2,6-linked sialyl groups. In certain embodiments, at least 50% of the sialyl groups of the N-glycans of the glycoproteins are α-2,6-linked sialyl groups. In general, terminal sialyl groups can be attached to the galactose groups via a α-2,3- or via a α-2,6-linkage. The N-glycans of the polypeptide of the invention may comprise more α-2,6-linked sialyl groups than α-2,3-linked sialyl groups. At least 60%, or at least 70%, or at least 80%, or at least 90% of the sialyl groups of the N-glycans may be α-2,6-linked sialyl groups. These embodiments can be obtained by using cell lines of human origin for expression of the polypeptide of the invention or, e.g., by co-expressing human α-2,6-sialyltransferase in mammalian cells.

Suitable methods of producing such glycoproteins are e.g. described in WO 2016/188905. Accordingly, a method of producing a glycoprotein comprising N-glycans with increased sialylation is described therein, which method comprises (i) providing cells comprising a nucleic acid encoding a polypeptide comprising a truncated von Willebrand Factor (VWF), and (ii) culturing said cells at a temperature of less than 36.0° C. In addition, a method of producing a dimer of a glycoprotein comprising a truncated von Willebrand Factor (VWF), or for increasing the dimerization of said glycoprotein is described, which method comprises (i) providing cells comprising a nucleic acid encoding the amino acid sequence of the glycoprotein, and (ii) culturing said cells at a temperature of less than 36.0° C. Further, a method of producing a glycoprotein comprising N-glycans with increased sialylation is described therein, which comprises (i) providing cells comprising a nucleic acid encoding a polypeptide comprising a truncated von Willebrand Factor (VWF) and a recombinant nucleic acid encoding an α-2,6-sialyltransferase, and (ii) culturing the cells under conditions that allow expression of the glycoprotein and of the α-2,6-sialyltransferase.

In one embodiment, at least 85%, at least 90%, at least 95%, at least 96%, at least 97%, at least 98%, or at least 99%, of the N-glycans of the polypeptide of the invention comprise at least one sialic acid group.

In another embodiment, less than 50%, or less than 25%, or less than 15%, or less than 12%, or less than 10%, or less than 8%, or less than 6%, or less than 5%, or less than 4%, or less than 3%, or less than 2% or even less than 1% of the N-glycans of the polypeptide of the invention are asialo-N-glycans, i.e. they are N-glycans lacking a sialic acid group.

Other embodiments of the invention comprise a truncated von Willebrand Factor (VWF), wherein said truncated VWF is capable of binding to a Factor VIII (FVIII), and wherein said glycoprotein comprises N-glycans, wherein less than 50%, preferably less than 40%, preferably less than 35%, preferably less than 30%, preferably less than 29%, preferably less than 28%, preferably less than 27% preferably less than 26%, preferably less than 25%, preferably less than 24%, preferably less than 23%, preferably less than 22%, preferably less than 21%, preferably less than 20%, preferably less than 19%, preferably less than 18%, preferably less than 17%, preferably less than 16%, preferably less than 15%, preferably less than 14%, preferably less than 13%, preferably less than 12%, preferably less than 11%, preferably less than 10%, preferably less than 9%, preferably less than 8%, preferably less than 7%, preferably less than 6% and preferably less than 5% of said N-glycans comprise, on average, two or more terminal and non-sialylated galactose residues.

Still other embodiments of the invention comprise a truncated von Willebrand Factor (VWF), wherein said truncated VWF is capable of binding to a Factor VIII (FVIII), and wherein said truncated VWF comprises N-glycans, wherein less than 20%, preferably less than 10%, preferably less than 5%, preferably less than 4%, preferably less than 3%, preferably less than 2%, and preferably less than 1% of said N-glycans comprise, on average, three or more terminal and non-sialylated galactose residues.

The above-described embodiments can be combined with each other. Any percentages of N-glycans mentioned above, or any indications of the degree of sialylation, are to be understood as average percentages or degrees, i.e. they refer to a population of molecules, not to a single molecule. It is clear that the glycosylation or sialylation of the individual glycoprotein molecules within a population of glycoproteins will show some heterogeneity.

Dimers

The polypeptides of this invention have a high proportion of dimers. The polypeptide of the invention is therefore preferably present as dimer. In one embodiment, at least 50%, or at least 60%, or at least 70%, or at least 80%, or at least 90%, or at least 95% or at least 98% of the polypeptides are present as dimers. In another embodiment, the ratio dimer:monomer of the polypeptide of the invention is at least 1.5, preferably at least 2, more preferably at least 3 or at least 5. Most preferably essentially all polypeptides of the invention are present as dimers. Further preferred is that the polypeptide of the invention does not comprise multimeric forms. The use of dimers is favorable, as the dimer has an improved affinity to Factor VIII as compared to the monomer. The dimer content and the ratio of dimer to monomer of the polypeptide of the invention can be determined as described in Example 2.

In one embodiment, the affinity of the polypeptide of the invention to Factor VIII is greater than that of human native VWF to the same Factor VIII molecule. The Factor VIII affinity of the polypeptide may refer to human native, either plasma-derived or recombinant, Factor VIII, in particular to a recombinant Factor VIII molecule having a truncated or deleted B-domain, preferably a Factor VIII molecule as characterized by SEQ ID NO:5.

It has been found that preparations of the polypeptide of this invention with a high proportion of dimers do have an increased affinity to Factor VIII. Alternatively to or in combination with an increased dimer proportion also polypeptides in accordance with the invention with mutations within the Factor VIII binding domain which do increase the affinity to Factor VIII are preferred embodiments of the invention. Suitable mutations are disclosed, e.g., in WO 2013/120939 A1.

Preparation of the Polypeptide

The nucleic acid encoding the polypeptide of the invention can be prepared according to methods known in the art. Based on the cDNA sequence of pre-pro form of human native VWF (SEQ ID NO:3), recombinant DNA encoding the above-mentioned truncated VWF constructs or polypeptides of the invention can be designed and generated.

Even if the polypeptide which is secreted by the host cells does not comprise amino acids 1 to 763 of pre-pro form of human VWF, it is preferred that the nucleic acid (e.g. the DNA) encoding the intracellular precursor of the polypeptide comprises a nucleotide sequence encoding an amino acid sequence having a sequence identity of at least 95%, at least 96%, at least 97%, at least 98%, or at least 99%, to amino acids 23 to 763 or preferably to amino acids 1 to 763 of SEQ ID NO:4. Most preferably, the nucleic acid (e.g. the DNA) encoding the intracellular precursor of the polypeptide comprises a nucleotide sequence encoding amino acids 23 to 763 of SEQ ID NO:4, or amino acids 1 to 763 of SEQ ID NO:4.

Constructs in which the DNA contains the entire open reading frame inserted in the correct orientation into an expression plasmid may be used for protein expression. Typical expression vectors contain promoters that direct the synthesis of large amounts of mRNA corresponding to the inserted nucleic acid in the plasmid-bearing cells. They may also include an origin of replication sequence allowing for their autonomous replication within the host organism, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Typically, the cells to be provided are obtained by introducing the nucleic acid encoding a polypeptide of the invention into mammalian host cells.

Any host cell susceptible to cell culture, and to expression of glycoproteins, may be utilized in accordance with the present invention. In certain embodiments, a host cell is mammalian. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); baby hamster kidney cells (BHK, ATCC CCL10); Chinese hamster ovary cells+/−DHFR (CHO, Urlaub and Chasin, Proc. Natl. Acad. Sci. USA, 77:4216, 1980); mouse sertoli cells (TM4, Mather, Biol. Reprod., 23:243 251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (HepG2, HB 8065); mouse mammary tumor (MMT 060562, ATCC CCL51); TRI cells (Mather et al., Annals NY. Acad. Sci., 383:44-68, 1982); MRC 5 cells; PS4 cells; human amniocyte cells (CAP); and a human hepatoma line (Hep G2). Preferably, the cell line is a rodent cell line, especially a hamster cell line such as CHO or BHK or a human cell line.

Methods suitable for introducing nucleic acids sufficient to achieve expression of a glycoprotein of interest into mammalian host cells are known in the art. See, for example, Gething et al., Nature, 293:620-625, 1981; Mantei et al., Nature, 281:40-46, 1979; Levinson et al. EP 117,060; and EP 117,058. For mammalian cells, common methods of introducing genetic material into mammalian cells include the calcium phosphate precipitation method of Graham and van der Erb (Virology, 52:456-457, 1978) or the Lipofectamine™ (Gibco BRL) Method of Hawley-Nelson (Focus 15:73, 1993). General aspects of mammalian cell host system transformations have been described by Axel in U.S. Pat. No. 4,399,216. For various techniques for introducing genetic material into mammalian cells, see Keown et al., Methods in Enzymology, 185:527-537, 1990, and Mansour et al., Nature, 336:348-352, 1988.

The cells are cultured under conditions that allow expression of the polypeptide. The polypeptide can be recovered and purified using methods that are known to the skilled artisan.

Factor VIII

As used herein, the term “Factor VIII” or “FVIII” refers to molecules having at least part of the coagulation activity of human native Factor VIII. Human FVIII consists of 2351 amino acids (including a signal peptide) and 2332 amino acids (without the signal peptide). “Human native FVIII” is the human plasma-derived FVIII molecule having the full length sequence as shown in SEQ ID NO:7 (amino acid 1-2332). The detailed domain structure, A1-a1-A2-a2-B-a3-A3-C1-C2 has the corresponding amino acid residues (referring to SEQ ID NO:7): A1 (1-336), a1 (337-372), A2 (373-710), a2 (711-740), B (741-1648), a3 (1649-1689), A3 (1690-2020), C1 (2021-2173) and C2 (2174-2332).

The coagulation activity of the FVIII molecule can be determined using a one-stage clotting assay (e.g. as described in Lee et al., Thrombosis Research 30, 511 519 (1983)) or a chromogenic substrate assay (e.g. the comatic FVIII test kit from Chromogenix-Instrumentation Laboratory SpA V. le Monza 338-20128 Milano, Italy). Further details of these activity assays are described infra.

Preferably, the FVIII molecules used in accordance with this invention have at least 10% of the specific molar activity of human native FVIII. The term “specific molar activity” refers to the coagulation activity per mole of FVIII and is indicated e.g. in “IU/mole” FVIII or—more convenient—in “IU/pmole” FVIII.

In a preferred embodiment, the FVIII molecule is a non-naturally occurring FVIII molecule. Preferably, the non-naturally occurring FVIII molecule has been produced recombinantly. In another embodiment, the FVIII molecule has been produced in cell culture. In another preferred embodiment, the non-naturally occurring FVIII molecule has a glycosylation pattern different from that of plasma-derived FVIII. In yet another embodiment, the FVIII molecule is selected from the group consisting of (i) B-domain deleted or truncated FVIII molecules, (ii) single-chain FVIII molecules, (iii) recombinantly produced two-chain FVIII molecules, (iv) FVIII molecules having protective groups or half-life extending moieties, (v) fusion proteins comprising a FVIII amino acid sequence fused to a heterologous amino acid sequence, and (vi) combinations thereof.

In another preferred embodiment, the FVIII molecule is a plasma-derived FVIII molecule.

The terms “Factor VIII” and “FVIII” are used synonymously herein. “Factor VIII compositions” in the sense of the present invention include compositions comprising FVIII and FVIIIa. FVIIIa may typically be present in small amounts, e.g. about 1 to 2% FVIIIa, referred to the total amount of FVIII protein in the composition. Proteolytically cleaved FVIII may typically be present in small to medium amounts, e.g. about 1 to 50%, referred to the total amount of FVIII protein in the composition. “FVIII” includes natural allelic variations of FVIII that may exist and occur from one individual to another. FVIII may be plasma-derived or recombinantly produced, using well-known methods of production and purification. The degree and location of glycosylation, tyrosine sulfation and other post-translation modifications may vary, depending on the chosen host cell and its growth conditions.

The term FVIII includes FVIII analogues. The term “FVIII analogue” as used herein refers to a FVIII molecule (full-length or B-domain-truncated/deleted) wherein one or more amino acids have been substituted or deleted compared to SEQ ID NO:7 or, for B-domain truncated/deleted FVII molecules, the corresponding part of SEQ ID NO:7. FVIII analogues do not occur in nature but are obtained by human manipulation.

The Factor VIII molecules included in the compositions of the present invention may also be B-domain-truncated/deleted FVIII molecules wherein the remaining domains correspond to the sequences as set forth in amino acid numbers 1-740 and 1649-2332 of SEQ ID NO:7. Other forms of B-domain deleted FVIII molecules have additionally a partial deletion in their a3 domain, which leads to single-chain FVIII molecules.

It follows that these FVIII molecules are recombinant molecules produced in transformed host cells, preferably of mammalian origin. However, the remaining domains in a B-domain deleted FVIII, (i.e. the three A-domains, the two C-domains and the a1, a2 and a3 regions) may differ slightly e.g. about 1%, 2%, 3%, 4% or 5% from the respective amino acid sequence as set forth in SEQ ID NO:7 (amino acids 1-740 and 1649-2332).

The FVIII molecules included in the composition of the present invention may be two-chain FVIII molecules or single-chain FVIII molecules. The FVIII molecules included in the composition of the present invention may also be biologically active fragments of FVIII, i.e., FVIII wherein domain(s) other than the B-domain has/have been deleted or truncated, but wherein the FVIII molecule in the deleted/truncated form retains its ability to support the formation of a blood clot. FVIII activity can be assessed in vitro using techniques well known in the art. A preferred test for determining FVIII activity according to this invention is the chromogenic substrate assay or the one stage assay (see infra). Amino acid modifications (substitutions, deletions, etc.) may be introduced in the remaining domains, e.g., in order to modify the binding capacity of Factor VIII with various other components such as e.g. Von Willebrand Factor (vWF), low density lipoprotein receptor-related protein (LPR), various receptors, other coagulation factors, cell surfaces, etc. or in order to introduce and/or abolish glycosylation sites, etc. Other mutations that do not abolish FVIII activity may also be accommodated in a FVIII molecule/analogue for use in a composition of the present invention.

FVIII analogues also include FVIII molecules, in which one or more of the amino acid residues of the parent polypeptide have been deleted or substituted with other amino acid residues, and/or wherein additional amino acid residues has been added to the parent FVIII polypeptide.

Furthermore, the Factor VIII molecules/analogues may comprise other modifications in e.g. the truncated B-domain and/or in one or more of the other domains of the molecules (“FVIII derivatives”). These other modifications may be in the form of various molecules conjugated to the Factor VIII molecule, such as e.g. polymeric compounds, peptidic compounds, fatty acid derived compounds, etc.

The term FVIII includes FVIII molecules having protective groups or half-life extending moieties. The terms “protective groups”/“half-life extending moieties” is herein understood to refer to one or more chemical groups attached to one or more amino acid site chain functionalities such as —SH, —OH, —COOH, —CONH2, —NH2, or one or more N- and/or O-glycan structures and that can increase in vivo circulatory half-life of a number of therapeutic proteins/peptides when conjugated to these proteins/peptides. Examples of protective groups/half-life extending moieties include: Biocompatible fatty acids and derivatives thereof, Hydroxy Alkyl Starch (HAS) e.g. Hydroxy Ethyl Starch (HES), Poly (Glyₓ-Ser_{y})ₙ (Homo Amino acid Polymer (HAP)), Hyaluronic acid (HA), Heparosan polymers (HEP), Phosphorylcholine-based polymers (PC polymer), Fleximer® polymers (Mersana Therapeutics, MA, USA), Dextran, Poly-sialic acids (PSA), polyethylene glycol (PEG), an Fc domain, Transferrin, Albumin, Elastin like peptides, XTEN® polymers (Amunix, CA, USA), Albumin binding peptides, a von Willebrand factor fragment (vWF fragment), a Carboxyl Terminal Peptide (CTP peptide, Prolor Biotech, IL), and any combination thereof (see, for example, McCormick, C. L., A. B. Lowe, and N. Ayres, Water-Soluble Polymers, in Encyclopedia of Polymer Science and Technology. 2002, John Wiley & Sons, Inc.). The manner of derivatization is not critical and can be elucidated from the above.

The term FVIII includes glyco-pegylated FVIII. In the present context, the term “glyco-pegylated FVIII” is intended to designate a Factor VIII molecule (including full length FVIII and B-domain truncated/deleted FVIII) wherein one or more PEG group(s) has/have been attached to the FVIII polypeptide via the polysaccharide sidechain(s) (glycan(s)) of the polypeptide.

The FVIII molecules which can be used in accordance with this invention include fusion proteins comprising a FVIII amino acid sequence fused to a heterologous amino acid sequence, preferably a half-life extending amino acid sequence. Preferred fusion proteins are Fc fusion proteins and albumin fusion proteins. The term “Fc fusion protein” is herein meant to encompass FVIII fused to an Fc domain that can be derived from any antibody isotype. An IgG Fc domain will often be preferred due to the relatively long circulatory half-life of IgG antibodies. The Fc domain may furthermore be modified in order to modulate certain effector functions such as e.g. complement binding and/or binding to certain Fc receptors. Fusion of FVIII with an Fc domain, which has the capacity to bind to FcRn receptors, will generally result in a prolonged circulatory half-life of the fusion protein compared to the half-life of the wt FVIII. It follows that a FVIII molecule for use in the present invention may also be a derivative of a FVIII analogue, such as, for example, a fusion protein of an FVIII analogue, a PEGylated or glycoPEGylated FVIII analogue, or a FVIII analogue conjugated to a heparosan polymer. The term “albumin fusion protein” is herein meant to encompass FVIII fused to an albumin amino acid sequence or a fragment or derivative thereof. The heterologous amino acid sequence may be fused to the N- or C-terminus of FVIII, or it may be inserted internally within the FVIII amino acid sequence. The heterologous amino acid sequence may be any “half life extending polypeptide” described in WO 2008/077616 A1, the disclosure of which is incorporated herein by reference.

Examples of FVIII molecules for use in compositions of the present invention comprise for instance the FVIII molecules described in WO 2010/045568, WO 2009/062100, WO 2010/014708, WO 2008/082669, WO 2007/126808, US 2010/0173831, US 2010/0173830, US 2010/0168391, US 2010/0113365, US 2010/0113364, WO 2003/031464, WO 2009/108806, WO 2010/102886, WO 2010/115866, WO 2011/101242, WO 2011/101284, WO 2011/101277, WO 2011/131510, WO 2012/007324, WO 2011/101267, WO 2013/083858, and WO 2004/067566.

Examples of FVIII molecules, which can be used in a composition of the present invention include the active ingredient of Advate®, Helixate®, Kogenate®, Xyntha®, Adynovate®, Kovaltry®, Nuwiq®, Novoeight®, Eloctate®, as well as the FVIII molecule described in WO 2008/135501, WO 2009/007451 and the construct designated “dBN(64-53)” of WO 2004/067566. This construct has the amino acid sequence shown in SEQ ID NO 5.

The concentration of Factor VIII in the composition of the present invention is typically in the range of 10-10,000 IU/mL. In different embodiments, the concentration of FVIII molecules in the compositions of the invention is in the range of 10-8,000 IU/mL, or 10-5,000 IU/mL, or 20-3,000 IU/mL, or 50-1,500 IU/mL, or 3,000 IU/mL, or 2,500 IU/mL, or 2,000 IU/mL, or 1,500 IU/mL, or 1,200 IU/mL, 1,000 IU/mL, or 800 IU/mL, or 600 IU/mL, or 500 IU/mL, or 400 IU/mL, or 300 IU/mL, or 250 IU/mL, or 200 IU/mL, or 150 IU/mL, or 100 IU/mL.

“International Unit,” or “IU,” is a unit of measurement of the blood coagulation activity (potency) of FVIII as measured by a FVIII activity assay such as a one stage clotting assay or a chromogenic substrate FVIII activity assay using a standard calibrated against an international standard preparation calibrated in “IU”. One stage clotting assays are known to the art, such as that described in N Lee, Martin L, et al., An Effect of Predilution on Potency Assays of FVIII Concentrates, Thrombosis Research (Pergamon Press Ltd.) 30, 511 519 (1983). Principle of the one stage assay: The test is executed as a modified version of the activated Partial Thromboplastin Time (aPTT)-assay: Incubation of plasma with phospholipids and a surface activator leads to the activation of factors of the intrinsic coagulation system. Addition of calcium ions triggers the coagulation cascade. The time to formation of a measurable fibrin clot is determined. The assay is executed in the presence of Factor VIII deficient plasma. The coagulation capability of the deficient plasma is restored by Coagulation Factor VIII included in the sample to be tested. The shortening of coagulation time is proportional to the amount of Factor VIII present in the sample. The activity of Coagulation Factor VIII is quantified by direct comparison to a standard preparation with a known activity of Factor VIII in International Units.

Another standard assay is a chromogenic substrate assay. Chromogenic substrate assays may be purchased commercially, such as the comatic FVIII test kit (Chromogenix-Instrumentation Laboratory SpA V. le Monza 338-20128 Milano, Italy). Principle of the chromogenic assay: In the presence of calcium and phospholipid, Factor X is activated by Factor IXa to Factor Xa. This reaction is stimulated by Factor VIIIa as cofactor. FVIIIa is formed by low amounts of thrombin in the reaction mixture from FVIII in the sample to be measured. When using the optimum concentrations of Ca²⁺, phospholipid and Factor IXa and an excess quantity of Factor X, activation of Factor X is proportional to the potency of Factor VIII. Activated Factor X releases the chromophore pNA from the chromogenic substrate S-2765. The release of pNA, measured at 405 nm, is therefore proportional to the amount of FXa formed, and, therefore, also to the Factor VIII activity of the sample.

Freeze-drying or lyophilization, unless otherwise indicated by the context in which it appears, shall be used to denote a drying process in which a solution of materials (i.e. an active pharmaceutical ingredient and various formulation additives or “excipients”) is converted into a solid. A typical freeze-drying process consists of three stages, “freezing”, “primary drying” and “secondary drying”. In the freezing stage almost all contained water is converted into ice and solutes into solids (crystalline or amorphous). In the primary drying stage the ice is removed from the product by direct sublimation which is achieved by maintaining a favorable pressure gradient between the water molecules (ice) and the surrounding atmosphere. In the secondary drying stage residual moisture is removed from the product by desorption.

If concentrations (w/v) are given for freeze-dried compositions they refer to the volume directly prior to freeze-drying.

Unless otherwise noted, percentage terms express weight/weight percentages and temperatures are in the Celsius scale.

Stability of FVIII

When co-formulated in one composition the polypeptide of the invention inhibits or reduces the loss in FVIII activity in the composition. The loss in FVIII activity of a FVIII formulation over time or during a specific processing step is lower than that of a control formulation lacking the polypeptide of the invention.

The term “control formulation” or “control composition”, as used herein, refers to a formulation which has the same constituents in the same amounts except that the polypeptide of the invention is missing. The loss in FVIII activity of (i) the composition comprising the polypeptide of the invention and the FVIII and of (ii) the control composition is measured (i) after storage for the identical period of time under identical conditions, and/or (ii) after subjecting both compositions to the identical processing steps. A processing step may be freeze-drying and optionally reconstitution.

In one embodiment, the polypeptide of the invention comprising a truncated VWF increases the stability of FVIII in a liquid composition. During storage in liquid form for one week at 25° C., the loss in FVIII activity in a composition comprising FVIII and the polypeptide of the invention is preferably less than 10%, more preferably less than 9%, most preferably less than 8%. During storage in liquid form for two weeks at 25° C., the loss in FVIII activity is preferably less than 15%. During storage in liquid form for three weeks at 25° C., the loss in FVIII activity is preferably less than 17%. During storage in liquid form for four weeks at 25° C., the loss in FVIII activity is preferably less than 20%. During storage in liquid form for six weeks at 25° C., the loss in FVIII activity is preferably less than 30%. The loss in FVIII activity can be determined as described in the examples of this application.

In another embodiment, the polypeptide of the invention comprising a truncated VWF increases the stability of FVIII during freeze-drying. The loss in FVIII activity upon freeze-drying a composition comprising FVIII and the polypeptide of the invention is preferably less than 15%, more preferably less than 10%, most preferably less than 5%.

In another embodiment, the polypeptide of the invention comprising a truncated VWF increases the stability of FVIII in a lyophilized composition. During storage in lyophilized form for 12 months at 25° C., the loss in FVIII activity in a composition comprising FVIII and the polypeptide of the invention is preferably less than 16%. During storage in lyophilized form for 18 months at 25° C., the loss in FVIII activity in a composition comprising FVIII and the polypeptide of the invention is preferably less than 25%. During storage in lyophilized form for 24 months at 25° C., the loss in FVIII activity in a composition comprising FVIII and the polypeptide of the invention is preferably less than 30%.

Pharmaceutical Compositions

The polypeptide comprising a truncated VWF as described herein may preferably be used for increasing the in vitro stability of coagulation factor VIII (FVIII) in a composition comprising said FVIII and said polypeptide. The composition is preferably a formulation.

The term “formulation”, as used herein, refers to a pharmaceutical formulation. The terms “pharmaceutical formulation” and “therapeutic formulation” are used synonymously herein if not indicated otherwise.

The formulation is preferably suitable for the treatment or prophylaxis of a blood coagulation disorder. The blood coagulation disorder is in particular hemophilia A.

Therapeutic formulations of the polypeptide of the invention can be prepared for storage as lyophilized formulations or aqueous solutions by mixing the polypeptide of the invention having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiologically acceptable conditions. They can present at concentration ranging typically from about 2 mM to about 100 mM. Suitable buffering agents include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to retard microbial growth, and can be added typically in amounts ranging from 0.2%-1% (w/v). Suitable preservatives include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

Tonicity modifying agents, sometimes also acting as “stabilizers”, can be added to ensure a pharmaceutically acceptable tonicity, preferably isotonicity, of liquid compositions and include inorganic salts such as sodium chloride and polyhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight polypeptide of the invention. Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include Pluronic polyols (polyoxamers 184, 188 etc.) and polyoxyethylene sorbitan monoethers (as e. g. polysorbates 20 and 80 etc.). Non-ionic surfactants can be present in a range of about 0.01 mg/ml to about 1.0 mg/ml, or in a range of about 0.05 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

In one embodiment the pharmaceutical composition described herein comprises an aqueous composition of the truncated von Willebrand Factor (VWF), said pharmaceutical composition further comprising:
a. a FVIII molecule;
b. 40 to 195 mM of a sodium salt;
c. histidine;
d. at least 1 mM of a calcium salt; and
e. a surfactant;

wherein [His]≥180 mM−20*[Ca²⁺], wherein [Ca²⁺] is the concentration of calcium ions in the aqueous composition in millimole per liter, and [His] is the concentration of histidine in the aqueous composition in millimole per liter, with the proviso that [His]>0; and wherein the osmolarity of the composition is 600 mOsmol/L or less. The concentration of the sodium salt in the aqueous composition may be between 45 to 95 mM. The concentration of histidine [His] can be between 5 to 200 mM. The composition may comprise calcium ions [Ca²⁺] with a concentration of between 5 to 100 mM. The composition may have a pH from 5 to 9. The calcium salt of the composition may preferably be calcium chloride or sodium chloride. The composition may further comprise a carbohydrate. The carbohydrate may preferably be sucrose. The concentration of the carbohydrate may be between 1 to 20% (w/w). The concentration of said surfactant may be between 0.001 to 0.2% (v/v). The surfactant may be a non-naturally occurring surfactant. The composition may further comprise at least one amino acid other than histidine. Said at least one amino acid other than histidine may be selected from the group consisting of arginine, asparagine, aspartic acid, glutamic acid, glutamine, lysine, methionine, phenylalanine, leucine, isoleucine and combinations thereof. The composition may further comprise at least one antioxidant, wherein said at least one antioxidant may be selected from the group consisting of reduced glutathione, methionine, cysteine, sodium sulfite, vitamin A, vitamin E, ascorbic acid, sodium ascorbate and combinations thereof. The concentration of said at least one antioxidant may vary between 0.05-100 mM.

In one embodiment the pharmaceutical composition described herein comprises in addition to the truncated von Willebrand Factor (VWF) sodium chloride, saccharose, L-arginine, calcium ions, a surfactant and citrate. Preferably, the composition further comprises Factor VIII. In a preferred embodiment, the pharmaceutical composition of the present invention comprises 10-30 mg/mL sodium chloride, 3-8 mg/mL saccharose, 3-8 mg/mL L-Arg×2H₂O, 0.1-0.5 mg/mL CaCl₂×2H₂O, 0.5-2 mg/mL Poloxamer 188, and 0.5-2 mg/mL Na-citrate×2H₂O. In a specific preferred embodiment the pharmaceutical composition of the present invention comprises 18 mg/mL sodium chloride, 5.4 mg/mL saccharose, 5.4 mg/mL L-Arg×2H₂O, 0.3 mg/mL CaCl₂×2H₂O, 1.2 mg/mL Poloxamer 188, and 1.2 mg/mL Na-citrate×2H₂O.

In a further embodiment the pharmaceutical composition described herein comprises in addition to the truncated von Willebrand Factor (VWF) a Factor VIII, wherein the composition comprises L-Histidine, NaCl, CaCl₂, Sucrose, Polysorbate 80.

In a further embodiment the pharmaceutical composition described herein comprises in addition to the truncated von Willebrand Factor (VWF) a Factor VIII, wherein the composition comprises 20 mM L-Histidine, 280 mM NaCl, 3.4 mM CaCl₂, 0.6% w/v Sucrose, 0.02% v/v Polysorbate 80, at pH 7.

The formulation herein can also contain a further therapeutic agent in addition to a polypeptide of the invention, or to FVIII and a polypeptide of the invention.

The nucleotide and amino acid sequences shown in the sequence listing are summarized in the Table 1.

TABLE 1

| SEQ | |
|---|---|
| ID NO: | Remarks |
| 1 | DNA sequence encoding a polypeptide comprising acids 1 to |
| | 1242 of human VWF, a glycine/serine linker and human |
| | albumin; nucleotide positions (nt): |
| | nt 1-6: EcoRI restriction enzyme cleavage site |
| | nt 32-3757: coding sequence for VWF amino acids 1 to 1242 |
| | nt 3758-3850: coding sequence for glycine/serine linker |
| | nt 3851-5608: coding sequence for human albumin |
| | nt 5609-5616: Notl restriction enzyme cleavage site |
| 2 | Amino acid sequence encoded by SEQ ID NO: 1 (mature form): |
| | amino acid positions (aa): |
| | aa 1-479: VWF D′D3 region (VWF amino acids 764-1242) |
| | aa 480-510: glycine/serine linker |
| | aa 511-1095: human albumin |
| 3 | DNA sequence encoding the pre-pro form of human native VWF |
| 4 | Amino acid sequence encoded by SEQ ID NO: 3 |
| 5 | Amino acid sequence of a single chain Factor VIII molecule |
| 6 | Amino acid sequence of mature human serum albumin |
| 7 | Amino acid sequence of human native FVIII |

Certain embodiments of the invention will now be described with reference to the following examples which are intended for the purpose of illustration only and are not intended to limit the scope of the generality hereinbefore described.

### EXAMPLES

Chromogenic FVIII:C Assay

The chromogenic FVIII:C assay was performed using the Comatic FVIII test kit (Chromogenix-Instrumentation Laboratory SpA V. le Monza 338-20128 Milano, Italy).

Principle of the assay: In the presence of calcium and phospholipid, Factor X is activated by Factor IXa to Factor Xa. This reaction is stimulated by Factor VIIIa as cofactor. F VIIIa is formed by low amounts of thrombin in the reaction mixture from F VIII in the sample to be measured. When using the optimum concentrations of Ca²⁺, phospholipid and Factor IXa and an excess quantity of Factor X, activation of Factor X is proportional to the potency of Factor VIII. Activated Factor X releases the chromophore pNA from the chromogenic substrate S-2765. The release of pNA, measured at 405 nm, is therefore proportional to the amount of FXa formed and, therefore, also to the Factor VIII activity of the sample. The assay was adapted to be performed on automated coagulation analyzers, either the Behring Coagulation Timer (BCT) or Behring Coagulation System (BCS), both from Siemens Healthcare Diagnostics GmbH, Ludwig-Erhard-Straße 12, 65760 Eschborn, Germany.

### Example 1

Generation of D′D3 Albumin Fusion Protein (D′D3-FP)

The expression cassette for D′D3-FP consisting of cDNA encoding VWF amino acids 1 to 1242, a glycine/serine linker and the cDNA of human albumin was prepared by custom gene synthesis (Eurofins Genomics, Ebersberg, Germany). Through flanking restriction sites (EcoRI, NotI) the expression cassette was excised from the cloning vector supplied and inserted into a plRESneo3 vector (BD Biosciences, Franklin Lakes, N.J., USA) linearized with EcoRI and NotI. The resulting expression plasmid contained nucleotide sequences encoding the VWF propeptide, D′ and D3 (VWF amino acids 1 to 1242 of SEQ ID NO:4) fused to the albumin coding sequence through a short linker coding sequence under CMV promoter control. The nucleotide sequence of the coding sequence is displayed as SEQ ID NO:1, the amino acid sequence of the mature D′D3-FP is shown as SEQ ID NO:2.

The expression plasmid was grown up in XL10 Gold (Agilent Technologies) and purified using standard protocols (Qiagen, Hilden, Germany).

CHO K1 cells were transfected using the Lipofectamine 2000 reagent (Invitrogen) and grown up in serum-free medium (CD-CHO, Invitrogen) in the presence of 500-1000 μg/ml Geneticin. An expression plasmid encoding PACE/furin (pFu-797) as described in WO 2007/144173 was cotransfected to maximize propeptide cleavage efficacy. Single cell derived clones were grown up and selected according to their D′D3-FP expression yield as quantified by an albumin specific enzyme immunoassay (see below). The cell line finally selected for D′D3-FP fermentation was called T2050-CL3.

Production of D′D3-FP was carried out in bioreactors applying a fermentation process in perfusion mode. The fermentation process for the production of D′D3-containing polypeptides started with the thaw of cell line T2050-CL3 followed by cell expansion in shake flasks and finally a fermentation process in perfusion mode using the Sartorius BioStat B-DCU 5 L bioreactor and the BioStat STR 50 L single-use bioreactors. The BioSeps 10 L or 200 L (Applikon), respectively, were used as cell retention devices. Cell culture media were either PowerCHO3 (Lonza BESP1204) with 8 mM L-glutamine and 1 μM CuSO₄ or ProCHO5 (Lonza BESP1072) with 10 mM L-glutamine and 1 μM CuSO₄.

The seed trains in shake flasks were performed at 37° C., 7.5% CO₂ at a shaker speed of 160 rpm.

The 5 L bioreactor was inoculated with a target VCD of 2.5×10⁵ cells/mL. The cells were cultivated in PowerCHO3 with 8 mM L-glutamine and 1 μM CuSO₄ at a temperature of +37.0° C., a pH of 7.00, and at 30% oxygen saturation. A temperature shift to +34.0° C. (evaluated range +31° C. to +35° C.) was performed after initial harvests from the bioreactor run at +37° C. had been taken. The pH was controlled using CO₂ sparged as acid and NaHCO₃ as base. The overlay air flow rate was set to 0.5 L/min. A ring sparger was used as a sparging unit. The agitation rate was 150 rpm with a 2fold pitch blade impeller in down pull mode.

The 50 L bioreactor was inoculated with a target VCD of 3.0×10⁵ cells/mL. The cells were cultivated in ProCHO5 medium with 10 mM L-glutamine and 1 μM CuSO₄ at a temperature of +37.0° C., a pH of 6.90, and at 30% oxygen saturation. A temperature shift to +34.0° C. was performed after the initial one or two harvests. PH control as above, the overlay air flow rate was set to 2 L/min. A micro sparger was used as a sparging unit. The agitation rate was 90 rpm with a 2fold pitch blade impeller in down pull mode.

The perfusion was initiated when the VCD in the bioreactor was ≥1.0×10⁶ cells/mL. The perfusion rate was set to 1.0 volume/volume/day. The BioSep was operated in back flush mode with 5 (10) minutes runtime and 10 seconds back flush at a power input of 7 (30) W (numbers in brackets refer to the 50 L bioreactor). The perfusate and the bleed were filtered inline and collected in bags over 48 hours at +2 to +8° C. The VCD was controlled by active bleeding using a turbidity probe using glucose consumption as parameter with a target of 2 g/L glucose. Harvest and bleed were filtered inline, the harvest system consisting of a disposable filter and disposable bag was changed every second day.

To prepare material for the freeze-drying and stability studies described below D′D3 albumin fusion protein harvests were purified by affinity and size exclusion chromatography. Briefly, the cell-free harvest from the bioreactor was concentrated 30-fold using a TFF system (e.g. Pall Centramate 500 S) with a 30 kDa membrane (e.g Pall Centramate OS030T12). That concentrate was spiked with NaCl and EDTA to a final concentration of 0.75 M NaCl and 5 mM EDTA and loaded overnight on a CaptureSelect Human Albumin column (Life Technologies) which was pre-equilibrated with 20 mM Tris buffer pH 7.4. After washing the column with equilibration buffer D′D3-FP was eluted with elution buffer (20 mM Tris, 2 M MgCl₂, pH 7.4). The eluate was then 10-fold concentrated and dialyzed against 50 mM Tris, 150 mM NaCl, pH 7.4 using Ultra Centrifugal Filters with a 30 kDa cut-off (e.g. Amicon. UFC903024). To separate the D′D3-FP dimer from the monomer portion that material was loaded on a Superdex 200 pg column (GE Healthcare Code: 17-1069-01) pre-equilibrated with 50 mM Tris, 150 mM NaCl, pH 7.4 and the peak fractions containing the D′D3-FP dimer were pooled. The area under the curve for the dimer and monomer peak fractions were used to calculate dimer to monomer ratio. Dimer preparations of D′D3-FP were used for the experiments in the following Examples.

rVIII-SingleChain

The examples were carried out using a FVIII molecule (construct dBN(64-53) described in WO 2004/067566). This FVIII molecule will be referred to as “rVIII-SingleChain” in the following.

### Example 2: Determination of FVIII Affinity to VWF Fragment Dimer and Monomer

A VWF fragment (1-1242) albumin fusion (D′D3-FP) containing a short linker sequence as described in Example 1 was expressed in a bioreactor; after purification as described above and isolation of monomer and dimer, the affinity of FVIII to these preparations was assessed through surface plasmon resonance via a Biacore instrument (T200, GE Healthcare).

An anti-albumin antibody (MA1-20124, Thermo Scientific) was covalently coupled via its N-terminus to an activated CM 3 chip by NHS (N-Hydroxysuccinimide) and EDC (Ethanolamine hydrochloride), both contained in the amine coupling kit (BR1000-50) from GE Healthcare. For immobilization 3 μg/mL of the antibody were diluted in sodium acetate buffer (10 mM, pH 5.0) and the antibody solution was flown over the chip for 7 min. at a flow rate of 10 μL/min. After the immobilization procedure non-coupled dextran filaments were saturated by flowing ethanolamine solution (1 M, pH 8.3) over the chip for 5 min (at a flow rate of 10 μL/min). The aim of saturating the flow cell was to minimize unspecific binding of the analytes to the chip. A reference flow cell was set up by saturating an empty flow cell with ethanolamine by using the same procedure as above.

Dimeric and monomeric D′D3-FP proteins, respectively, were immobilized to the covalently coupled anti-albumin antibody by a flow of the D′D3-FP proteins (5 μg/mL) over the chip for 3 min (flow rate of 10 μL/min).

To create binding curves for FVIII, each D′D3-FP protein preparation was diluted in running buffer (HBS-P+: 0.1 M HEPES, 1.5 M NaCl and 0.5% v/v Surfactant P20, pH 7.4; product code BR100671, GE Healthcare) to concentrations of 0.25 nM, 0.5 nM, 1 nM, 3 nM and 4 nM. By performing a single cycle kinetic, samples with ascending concentrations of each dilution were flown over the chip for 2 min (flow rate 30 μL/min.), followed by a dissociation time of 10 min. with running buffer HBS-P+. All measurements were performed twice. The temperature for the measuring procedure was adjusted to +25° C.

Binding parameters were calculated using BiaEvaluation Software. The curve fitting methods were based on Langmuir equations. The input data for calculations were the molar mass of the analyte FVIII (rVIII-SingleChain), other parameters like max. RU and slopes were automatically extracted out of the fitted association and dissociation curves. The outputs of BiaEvaluation Software are the association rate constants and the dissociation rate constants, from which the affinity constants were calculated. The results are shown in Table 2.

TABLE 2
rVIII-SingleChain affinity data for D′D3-FP dimer and monomer

| D′D3-FP preparation | ka [1/Ms] | kd [1/s] | K_{D} [M] |
|---|---|---|---|
| D′D3-FP Dimer | 4.5 × 10⁷ | 1.5 × 10⁻³ | 3.4 × 10⁻¹¹ |
| D′D3-FP Monomer | 9.9 × 10⁵ | 3.0 × 10⁻² | 3.0 × 10⁻⁸ |

The dimeric D′D3-FP shows a significantly (K_{D}=34 μM) increased affinity to FVIII compared to the D′D3-FP monomer (K_{D}=30 nM) which results both from a faster association and a slower dissociation of rVIII-SingleChain.

### Example 3

Preparation of FVIII+rD′D3-FP formulations and evaluation of the FVIII:C recovery (i.e. activity by chromogenic assay) upon storage in liquid state.

Purified rVIII-SingleChain was formulated into the desired buffer & excipient composition (i.e. 20 mM L-Histidine, 280 mM NaCl, 3.4 mM CaCl₂, 0.6% w/v Sucrose, 0.02% v/v Polysorbate 80, pH 7) by preparative size exclusion chromatography (Superdex 200 pg; GE Healthcare, Ref. No. 17-1043). Purified rD′D3-FP was formulated into the same buffer & excipient matrix by dialysis. Both components were mixed together to achieve the desired FVIII activity and rD′D3-FP concentration as described in table 3. Prior to storage, the formulations were sterile filtered (Millex GV disposable filter units; Millipore, Ref. No. SLGV013SL) in order to avoid potential degradation induced by microbial contamination.

The different compositions were then investigated for their recovery in FVIII:C over a period of 6 weeks at 25° C.

The recovery (yield) of FVIII:C was calculated as the percentage of the amount of FVIII:C in the respective formulations following storage divided by the amount of FVIII:C in the solution prior to storage.

TABLE 3
Stability of rVIII-SingleChain in absence and presence of rD′D3-FP in the liquid state

| | Stability: FVIII:C Coamatic Recovery [%] | | | | | | |
|---|---|---|---|---|---|---|---|
| | +25° C. | | | | | | |
| | | 1 | 2 | 3 | 4 | 5 | 6 |
| Formulation | Time 0 | week | weeks | weeks | weeks | weeks | weeks |
| Control w/o rD′D3-FP | 100 | 87 | 83 | 83 | 78 | 71 | 68 |
| 100 IU/mL FVIII:C + 1.5 mg/mL rD′D3-FP | 100 | 93 | 87 | 85 | 89 | 73 | 74 |

### Example 4

Preparation of FVIII+rD′D3-FP formulations and evaluation of the FVIII:C recovery (i.e. activity by chromogenic assay) upon freeze-drying.

Purified rVIII-SingleChain was formulated into a buffer & excipient composition (20 mM L-Histidine, 280 mM NaCl, 3.4 mM CaCl₂, 0.6% w/v Sucrose, 0.02% v/v Polysorbate 80, pH 7) by preparative size exclusion chromatography (Superdex 200 pg). Purified rD′D3-FP was formulated into the same buffer & excipient matrix. Both components were mixed together to achieve the desired FVIII activity and rD′D3-FP concentration as described in table 4. The formulations were then dispensed (2.5 mL) and freeze-dried. The different compositions were then investigated for their loss in FVIII:C over freeze-drying.

The % loss of FVIII:C was calculated as the amount of FVIII:C in the reconstituted lyophilisates multiplied by 100 and divided by the amount of FVIII:C in the solution prior to freeze-drying subtracted from 100:

Loss of FVIII:C (%)=100−(100*FVIII:C after lyo/FVIII:C prior to lyo)

TABLE 4
Loss of FVIII:C upon freeze-drying depending
on the amounts of rD′D3-FP contained

| FVIII:C and rD′D3-FP content of | FVIII:C |
|---|---|
| Formulations prior to freeze-drying | [% loss on freeze-drying] |
| 100 IU/mL FVIII:C | 16 |
| Control w/o rD′D3-FP | |
| 100 IU/mL FVIII:C + | 13 |
| 0.015 mg/mL rD′D3-FP | |
| 100 IU/mL FVIII:C + | 10 |
| 0.150 mg/mL rD′D3-FP | |
| 100 IU/mL FVIII:C + | 11 |
| 0.375 mg/mL rD′D3-FP | |
| 100 IU/mL FVIII:C + | 5 |
| 1.5 mg/mL rD′D3-FP | |
| 100 IU/mL FVIII:C + | 2 |
| 4.5 mg/mL rD'D3-FP | |

### Example 5

Preparation of FVIII+rD′D3-FP formulations and evaluation of the FVIII:C recovery (i.e. activity by chromogenic assay) in freeze-dried samples upon long-term storage at elevated temperature.

Purified rVIII-SingleChain was formulated into a buffer & excipient composition (20 mM L-Histidine, 280 mM NaCl, 3.4 mM CaCl₂, 0.6% w/v Sucrose, 0.02% v/v Polysorbate 80, pH 7) by preparative size exclusion chromatography (Superdex 200 pg). Purified rD′D3-FP was formulated into the same buffer & excipient matrix. Both components were mixed together to achieve the desired FVIII activity and rD′D3-FP concentration as described in table 5. The formulations were then dispensed (2.5 mL) into vials and freeze-dried. The different compositions were then investigated for their recovery in FVIII:C after freeze-drying and after storage at +25° C. for up to 24 months.

The FVIII:C recovery (stability) was calculated as the percentage of FVIII:C still measurable after storage. The FVIII:C determined after freeze-drying and prior to storage was used as reference for the calculation.

TABLE 5
Storage stability of freeze-dried FVIII in absence or presence of different amounts of rD'D3-FP.

| | Storage stability of lyophilisates: FVIII:C Recovery [%] | | | | | | | |
|---|---|---|---|---|---|---|---|---|
| | +25° C. | | | | | | | |
| | after FD | 1 | 3 | 6 | 9 | 12 | 18 | 24 |
| | (T 0) | month | months | months | months | months | months | months |
| Control w/o rD′D3-FP | 100 | 107 | 102 | 94 | 87 | 83 | 73 | 70 |
| 100 IU/mL FVIII:C + 0.015 mg/mL rD′D3-FP | 100 | 95 | 98 | 83 | 81 | 77 | 67 | 62 |
| 100 IU/mL FVIII:C + 0.150 mg/mL rD′D3-FP | 100 | 99 | 98 | 84 | 79 | 78 | 67 | 62 |
| 100 IU/mL FVIII:C + 0.375 mg/mL rD′D3-FP | 100 | 106 | 106 | 91 | 94 | 85 | 79 | 71 |
| 100 IU/mL FVIII:C + 1.5 mg/mL rD′D3-FP | 100 | 104 | 98 | 88 | 84 | 87 | 81 | 74 |
| 100 IU/mL FVIII:C + 4.5 mg/mL rD′D3-FP | 100 | 106 | 101 | 91 | 95 | 92 | 87 | 82 |

Molar Ratios

Any molar ratios according to the invention refer to a ratio of the molar concentration of the (monomeric) subunit of the D′D3 containing polypeptide used in the invention, whether actually present as monomer or dimer, and of FVIII. More specifically, any ratios of rD′D3-FP over FVIII in this application refer to the amount or concentration of rD′D3-FP in the solution or in a freeze-dried product (in mole or mole/L) divided by the amount or concentration of FVIII in the solution or in a freeze-dried product (in mole or mole/L), unless indicated otherwise.

Typically, the concentration of FVIII is measured by activity testing (FVIII:C) and will be given in IU/mL. The concentration of rD′D3-FP (The recombinant fusion protein of the D′D3 domain of VWF with albumin) typically will be given in mg/mL. These values can be converted into molarities as described in the following.

The molecular weight of the monomeric subunit of rD′D3-FP (including glycosylation) used for calculation is 127,000 Da. The molecular weight of FVIII (rVIII-SingleChain) used for calculation (including glycosylation) is 180,000 Da and its specific activity used for calculation is 11,000 IU/mg.

TABLE 6
Calculation of molar ratios of several formulations
based on rD′D3-FP as D′D3 containing polypeptide
and rVIII-SingleChain as FVIII molecule.

| | | rD′D3-FP* | rD′D3-FP* | Molar ratio |
|---|---|---|---|---|
| FVIII conc. | FVIII conc. | conc. | conc. | rD′D3- |
| (IU/mL) | (nmol/L) | (mg/mL) | (nmol/L) | FP*/FVIII |
| 100 | 50.5 | 0.015 | 118 | 2.3 |
| 100 | 50.5 | 0.15 | 1181 | 23 |
| 100 | 50.5 | 0.375 | 2953 | 58 |
| 100 | 50.5 | 1.5 | 11810 | 234 |
| 100 | 50.5 | 4.5 | 35430 | 702 |

*basis for calculation was the monomer subunit

1. A method for increasing the in vitro stability of coagulation factor VIII (FVIII) in a composition, said method comprising:
mixing a polypeptide comprising a truncated von Willebrand Factor (VWF) and said FVIII to form a composition comprising the FVIII and the polypeptide comprising said truncated VWF, wherein the molar ratio of the polypeptide to the FVIII in the composition is greater than 20.

2. The method according to claim 1, wherein said polypeptide increases the storage stability of said FVIII.

3. The method according to claim 1, wherein the molar ratio of the polypeptide to the FVIII in the composition is greater than 50.

4. The method of claim 1, wherein the composition does not comprise wild-type VWF.

5. The method of claim 1, wherein said FVIII is either recombinantly produced FVIII or is a plasma derived FVIII.

6. The method of claim 1, wherein said method comprises adding a more than 20-fold molar excess to less than 10,000 fold excess of said polypeptide to said FVIII, thereby stabilizing said FVIII.

7. The method of claim 1, wherein the yield of FVIII upon freeze-drying and reconstituting the composition comprising the FVIII and the polypeptide is greater than the yield of FVIII upon freeze-drying and reconstituting a control composition lacking said polypeptide.

8. The method of claim 7, wherein the freeze-dried composition is reconstituted immediately after freeze-drying.

9. The method of claim 1, wherein the loss in FVIII activity during storage at 25° C. of a freeze-dried composition comprising the FVIII and the polypeptide is lower than that of a freeze-dried control composition lacking said polypeptide.

10. The method of claim 9, wherein said storage is for a period of 12 months.

11. The method according to claim 1, wherein the FVIII activity in a liquid composition comprising said polypeptide and said FVIII after storage at 25° C. for one week is greater than that of a control composition lacking said polypeptide.

12. The method according to claim 1, wherein said polypeptide binds to said FVIII with a dissociation constant K_{D} of 1 nM or less.

13. The method according to claim 1, wherein the truncated VWF comprises amino acids 764 to 1242 SEQ ID NO:4.

14. The method according to claim 1, wherein the truncated VWF lacks amino acids 1243 to 2813 of SEQ ID NO:4.

15. The method according to claim 1, wherein the truncated VWF consists of
(a) amino acids 764 to 1242 of SEQ ID NO:4,
(b) an amino acid sequence having a sequence identity of at least 90% to amino acids 764 to 1242 of SEQ ID NO:4, or
(c) a fragment of (a) or (b).

16. The method according to claim 1, wherein the truncated VWF does not comprise the binding sites for platelet glycoprotein Ibα (GPIbα), collagen, integrin αIIbβIII (RGDS sequence within the C1 domain) and/or the cleavage site (Tyr1605-Met1606) for ADAMTS13.

17. The method according to claim 1, wherein the polypeptide has low or essentially no affinity for platelets via GPIbα, said low or essentially no affinity being characterized by a dissociation constant K_{D}>1 μM, preferentially K_{D}>10 μM, for binding of the polypeptide to GPIbα.

18. The method according to claim 1, wherein the polypeptide does not contain the VWF domains A1 and/or A3 or a part thereof and does have low or essentially no affinity for collagen type I and type III, said low or essentially no affinity being characterized by a dissociation constant K_{D}>1 μM, preferentially K_{D}>10 μM, for binding of the polypeptide to collagen type I and type III).

19. The method according to claim 1, wherein the polypeptide is a fusion protein comprising the truncated VWF and a half-life extending polypeptide.

20. The method according to claim 19, wherein the half-life extending polypeptide is albumin or a fragment thereof.

21. The method according to claim 1, wherein the composition is a formulation.

22. A method for making a composition comprising coagulation factor VIII (FVIII) having increased in vitro stability, said method comprising:
mixing a polypeptide comprising a truncated von Willebrand Factor (VWF) and the FVIII to form a composition comprising the FVIII and the polypeptide comprising a truncated VWF, wherein the molar ratio of the polypeptide to the polypeptide comprising a truncated FVIII in the composition is greater than 20.

23. A method of storing a pharmaceutical composition comprising coagulation factor VIII (FVIII) and a truncated von Willebrand Factor (VWF), said method comprising:
storing the composition for a time period of at least one week, wherein the molar ratio of the polypeptide to the FVIII in the composition is greater than 20.

24. The method of claim 23, wherein the composition is in a freeze-dried form.

25. The method of claim 23, wherein the composition is stored at a temperature of 25° C.


SEQUENCE LISTING (7 sequences)

SEQ ID NO: 1 - DNA, 5616 bp, Artificial Sequence
  DNA encoding construct VWF fragment - G/S linker - albumin
  misc_feature (1)..(6): EcoRI restriction enzyme cleavage site
  misc_feature (32)..(3757): coding sequence for VWF amino acids 1 to 1242
  misc_feature (3758)..(3850): coding sequence for glycine/serine linker
  misc_feature (3851)..(5608): coding sequence for human albumin
  misc_feature (5609)..(5616): NotI restriction enzyme cleavage site
gaattcccgcagccctcatttgcaggggaagatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggctcgagcgggggatctggcgggtctggaggctctggagggtcgggaggctctggaggctctgggggatctggcgggtctggagggtcgggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataggcggccgc

SEQ ID NO: 2 - protein, 1096 aa, Artificial Sequence
  polypeptide encoded by SEQ ID NO1
  MISC_FEATURE (1)..(479): VWF D?D3 region (VWF amino acids 764 - 1242)
  MISC_FEATURE (1)..(100): Xaa is any amino acid
  MISC_FEATURE (480)..(510): glycine/serine linker
  MISC_FEATURE (511)..(1095): human albumin
XSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGSSGGSGGSGGSGGSGGSGGSGGSGGSGGSGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 3 - DNA, 8442 bp, Homo sapiens
  CDS (1)..(8442)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggggcgcgcccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtttataaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 4 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 5 - protein, 1444 aa, Artificial Sequence
  amino acid sequence of a single chain factor viii molecule
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 585 aa, Homo sapiens [sequence omitted]

SEQ ID NO: 7 - protein, 2332 aa, homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHRSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY